(19) United States
(12) Patent Application Publication
(10) Pub. No.: US 2024/0350769 A1
(43) Pub. Date: Oct. 24, 2024

(54) METERED CHEMICAL DOSE FOR RENAL DENERVATION

(71) Applicant: Medtronic Ireland Manufacturing Unlimited Company, Dublin 2 (IE)

(72) Inventor: Enda Carter, Galway (IE)

(21) Appl. No.: 18/681,378

(22) PCT Filed: Aug. 25, 2022
(86) PCT No.: PCT/EP2022/073690

§ 371 Date: Feb. 5, 2024

Related U.S. Application Data
(60) Provisional application No. 63/237,455, filed on Aug. 26, 2021.

(51) Int. Cl. A61M 25/00; A61M 5/28

(52) U.S. Cl. CPC A61M 25/0082; A61M 5/28

(57) ABSTRACT

A catheter (102) includes an elongate body (108) extending from a proximal end to a distal end, a distal portion (108b), and a therapeutic assembly (112) at the distal portion of the elongate body, wherein the therapeutic assembly comprises a chemical chamber configured to supply a predetermined volume of chemical agent to tissue adjacent the therapeutic assembly.

### TECHNICAL FIELD

The present technology is related to percutaneous neuromodulation systems. In particular, various examples of the present technology are related to percutaneous neuromodulation systems for renal neuromodulation.

### BACKGROUND

The sympathetic nervous system (SNS) is a primarily involuntary bodily control system typically associated with stress responses. Fibers of the SNS extend through tissue in almost every organ system of the human body and can affect characteristics such as pupil diameter, gut motility, and urinary output. Such regulation can have adaptive utility in maintaining homeostasis or in preparing the body for rapid response to environmental factors. Chronic over-activation of the SNS, however, is a common maladaptive response that can drive the progression of many disease states. Excessive activation of the renal SNS in particular has been identified experimentally and in humans as a likely contributor to the complex pathophysiology of arrhythmias, hypertension, states of volume overload (e.g., heart failure), and progressive renal disease.

Sympathetic nerves of the kidneys terminate in the renal blood vessels, the juxtaglomerular apparatus, and the renal tubules, among other structures. Stimulation of the renal sympathetic nerves can cause, for example, increased renin release, increased sodium reabsorption, and reduced renal blood flow. These and other neural-regulated components of renal function can be considerably stimulated in disease states characterized by heightened sympathetic tone. For example, reduced renal blood flow and glomerular filtration rate as a result of renal sympathetic efferent stimulation may be a cornerstone of the loss of renal function in cardio-renal syndrome, (i.e., renal dysfunction as a progressive complication of chronic heart failure). Pharmacologic strategies to thwart the consequences of renal sympathetic stimulation include centrally-acting sympatholytic drugs, beta blockers (e.g., to reduce renin release), angiotensin-converting enzyme inhibitors and receptor blockers (e.g., to block the action of angiotensin II and aldosterone activation consequent to renin release), and diuretics (e.g., to counter the renal sympathetic mediated sodium and water retention). These pharmacologic strategies, however, can have significant limitations including limited efficacy, compliance issues, side effects, and others.

### SUMMARY

The present technology is directed to devices, systems, and methods for supplying a specific volume or dose of chemical agent to a target site, such as to renal sympathetic nerves for renal denervation.

In some examples, a catheter comprises an elongate body extending from a proximal end to a distal end including a distal portion, and a therapeutic assembly at the distal portion of the elongate body, wherein the therapeutic assembly comprises a chemical chamber configured to supply a predetermined volume of chemical agent to tissue adjacent the therapeutic assembly.

In some examples, a method comprises positioning a distal portion of an elongate body of a catheter, including a therapeutic assembly, adjacent tissue at a treatment site within a patient, and supplying, via a chemical chamber of the therapeutic assembly, a predetermined volume of chemical agent to the tissue, wherein the chemical chamber contains the predetermined volume of chemical agent.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Reference is made to the attached drawings, wherein elements having the same reference numeral designations represent similar elements throughout:

FIG. 1 is a partially schematic illustration of a neuromodulation system configured in accordance with some examples of the present disclosure.

FIG. 2 illustrates accessing of a renal artery and modulating renal nerves with the system of FIG. 1 in accordance with some examples of the present disclosure.

FIG. 3 is a conceptual illustration of an example sympathetic nervous system (SNS) and how the brain communicates with the body via the SNS.

FIG. 4 is an enlarged anatomic view of nerves innervating a left kidney to form the renal plexus surrounding the left renal artery.

FIGS. 5 and 6 are anatomic and conceptual views, respectively, of a human body depicting neural efferent and afferent communication between the brain and kidneys.

FIGS. 7 and 8 are anatomic views of the arterial vasculature and venous vasculature, respectively, of a human.

FIG. 9A is a cross-section view conceptual diagram of a catheter and an example compliant chemical chamber in an uncompressed state, in accordance with some examples of the disclosure.

FIG. 9B is a side view conceptual diagram of the catheter and example compliant chemical chamber in the uncompressed state of FIG. 9A, in accordance with some examples of the disclosure.

FIG. 9C is a cross-section view conceptual diagram of a catheter and an example compliant chemical chamber of FIG. 9A in a partially compressed state, in accordance with some examples of the disclosure.

FIG. 9D is a side view conceptual diagram of the catheter and example compliant chemical chamber in the partially compressed state of FIG. 9C, in accordance with some examples of the disclosure.

FIG. 9E is a cross-section view conceptual diagram of a catheter and an example compliant chemical chamber of FIG. 9A in another compressed state, in accordance with some examples of the disclosure.

FIG. 9F is a side view conceptual diagram of the catheter and example compliant chemical chamber in the compressed state of FIG. 9E, in accordance with some examples of the disclosure.

FIG. 10A is a cross-section view conceptual diagram of a catheter and another example compliant chemical chamber in an uncompressed state, in accordance with some examples of the disclosure.

FIG. 10B is a side view conceptual diagram of the catheter and example compliant chemical chamber in the uncompressed state of FIG. 10A, in accordance with some examples of the disclosure.

FIG. 10C is a cross-section view conceptual diagram of a catheter and the example compliant chemical chamber of FIG. 10A in a partially compressed state, in accordance with some examples of the disclosure.

FIG. 10D is a side view conceptual diagram of the catheter and example compliant chemical chamber in the partially compressed state of FIG. 10C, in accordance with some examples of the disclosure.

FIG. 10E is a cross-section view conceptual diagram of a catheter and an example compliant chemical chamber of FIG. 10A in another compressed state, in accordance with some examples of the disclosure.

FIG. 10F is a side view conceptual diagram of the catheter and example compliant chemical chamber in the compressed state of FIG. 10E, in accordance with some examples of the disclosure.

FIGS. 11A and 11B are side view conceptual diagrams of an example compliant chemical chamber, including a ring, in two different phases of compression, in accordance with some examples of the disclosure.

FIGS. 12A and 12B are side view conceptual diagrams of an example compliant chemical chamber, including a ball, in two different phases of compression, in accordance with some examples of the disclosure.

FIGS. 13A, 13B, and 13C are side view conceptual diagrams of an example syringe system in three different phases of compression, including a gel bolus of defined volume and a plug, in accordance with some examples of the disclosure.

FIG. 14 is a flow diagram illustrating an example method for supplying a predetermined volume of chemical agent to a treatment site (i.e., patient tissue).

### DETAILED DESCRIPTION

The present disclosure is directed to devices, systems, and methods for neuromodulation, such as renal neuromodulation, using chemical agents.

As used herein, the terms “distal” and “proximal” define a position or direction with respect to the treating clinician or clinician's control device (e.g., an injection assembly). “Distal” or “distally” can refer to a position distant from or in a direction away from the clinician or clinician's control device. “Proximal” and “proximally” can refer to a position near or in a direction toward the clinician or clinician's control device.

Various medical ablation procedures may involve accessing and ablating tissues, such as nerves, near vasculature of a patient. As one example, renal neuromodulation may be used to treat a variety of conditions, such as hypertension, heart failure, or chronic kidney disease, by modulating activation of the renal sympathetic neural system. Renal sympathetic nerves of the renal sympathetic nervous system generally are near or within walls of renal arteries, such that the renal arteries may provide access to the renal sympathetic nerves. Renal neuromodulation treatments, such as renal denervation, may be accomplished using one or more of a variety of treatment modalities, including radio frequency (RF) energy, microwave energy, ultrasound energy, a chemical agent, or the like.

Renal neuromodulation using a chemical agent may be useful for ablating the renal sympathetic nerves. Chemical ablation may operate by injecting a chemical agent into tissues near the renal artery to chemically ablate the renal sympathetic nerves. The chemical agent may be selected to modulate activity of one or more renal nerves adjacent to the renal artery in which the neuromodulation catheter is positioned. For example, the chemical agent may be a neurotoxic chemical selected to chemically ablate the one or more renal nerves near the renal artery. During a chemical ablation procedure, a clinician may anesthetize a patient and guide a neuromodulation catheter through the patient's vasculature to a renal artery and a treatment site. The neuromodulation catheter may include at least one port or needle through which a chemical agent is delivered. Once the clinician has injected the chemical agent into the tissues near the renal artery and removed the neuromodulation catheter from the patient, the clinician may continue to monitor the patient over a span of days or weeks to determine whether the treated condition has been alleviated. In some examples, the patient may undergo additional iterations of this procedure until the condition has been sufficiently treated.

It may be desirable to inject a specific volume of chemical agent into tissues near the renal artery to chemically ablate the renal sympathetic nerves. Too little injected chemical agent may not achieve the desired neuromodulation, while too much injected chemical agent may damage unintended tissues. Some example chemical neuromodulation systems preload a catheter with a specific volume of chemical agent, as well as saline. If a clinician injects more fluid than the specific volume of chemical agent, saline enters the patient's body instead of excess chemical agent, so as to avoid excess damage to unintended tissues. However, saline can also dilute the chemical agent at the treatment site, reducing a likelihood of or even preventing effective neuromodulation.

In accordance with techniques of this disclosure, a catheter may include a therapeutic assembly with a chemical chamber configured to supply a predetermined volume of fluid (e.g., a predetermined dose of chemical agent) to tissue at a treatment site in a patient. The catheter includes an elongate body extending from a proximal end to a distal end and including at least a proximal portion and a distal portion. The therapeutic assembly that includes the chemical chamber may be positioned at the distal portion of the elongate body, near the port or needle through which the chemical agent enters the treatment site. By supplying a predetermined volume of chemical agent for neuromodulation, the present disclosure achieves a desired neuromodulation without excess delivery of chemical agent or delivery of another fluid that may affect operation of the chemical agent.

In some examples, the chemical chamber exclusively contains the predetermined volume of chemical agent (e.g., includes only the predetermined volume of chemical agent and does not include another fluid, such as saline, for example for pushing the chemical agent from the catheter). One or more walls of the chemical chamber may be compliant, such that the one or more walls deform when acted upon by a force. The force may be transmitted by a mechanical element, a hydraulic (e.g., fluid) element in the form of a fluid pressure, or the like. A clinician may apply a force or pressure to the chemical chamber, compressibly deforming the walls of the chemical chamber, and increasing fluid pressure within the chemical chamber and urging the chemical agent through an opening of the chemical chamber. The opening of the chemical chamber may be in fluid communication with a lumen defined by the port or needle through which the chemical agent exits the catheter and is delivered to the treatment site (e.g., is injected to the patient's tissue or released into the vessel in which the catheter is positioned). Once the chemical chamber is fully deformed, the predetermined volume of chemical agent has passed through the opening and into the treatment site through the port or needle, and no further fluid exits the chemical chamber into the treatment site. In some examples, the predetermined volume (dose) includes substantially all of the chemical agent within the chemical chamber, such that substantially all (e.g., all or nearly all) of the chemical agent is urged from the chemical chamber.

In some examples, a chemical chamber contains at least the predetermined volume of chemical agent, and the therapeutic assembly includes one or more features configured to stop chemical agent delivery upon release of the predetermined volume of chemical agent. For example, the therapeutic assembly may include a push member (such as a rigid rod) attached to a compression element configured to apply a compression or squeezing force or pressure to one or more compliant walls of the chemical chamber. The chemical chamber and compression element may be positioned next to one another inside the distal portion of the elongate body of the catheter. The distal portion of the elongate body may include a relatively large inner diameter portion and a relatively reduced inner diameter portion, wherein the inner diameter of the relatively reduced inner diameter portion is smaller than an inner diameter of the relatively large inner diameter portion, and wherein the relatively reduced inner diameter portion is positioned distal to the relatively large inner diameter portion. The push member may extend from the proximal end to the distal end of the elongate body, such that a clinician may apply a force or pressure to the compression element through manipulation of the push member. As the clinician advances the push member distally, the compression member advances distally from the relatively large inner diameter portion to the relatively reduced inner diameter portion, deforming the compliant wall of the chemical chamber, and increasing fluid pressure within the chemical chamber, and pushing the chemical agent through an opening of the chemical chamber. The relatively reduced inner diameter portion may include a specific length such that the volume of the chemical chamber positioned within the length is equal to the predetermined volume of chemical agent. Once the compression element has advanced through the relatively reduced inner diameter portion, the predetermined volume of chemical agent has passed through the opening and to the treatment site through the port or needle, and no further fluid enters the treatment site without first distally retracting the compression element and push member. In some examples, the predetermined volume (dose) includes less than all of the chemical agent within the chemical chamber, such that less than all of the chemical agent is urged from the chemical chamber when dispensing the dose.

In some examples, a chemical chamber contains at least the predetermined volume of chemical agent, and the therapeutic assembly includes a plug slightly larger than the opening, wherein the plug is configured to travel with the chemical agent towards the opening when the chemical agent is acted upon by the force or pressure. The plug may be positioned inside the chemical chamber a length away from the opening such that, as the plug travels along the length towards the opening when the chemical agent is acted upon by the force or pressure, the predetermined volume of chemical agent exits the chemical chamber before the plug reaches the opening and the plug prevents more fluid from exiting.

In some examples, the chemical chamber is sealed at both a proximal and a distal end, and the chemical chamber contains only the predetermined volume of chemical agent. When the chemical chamber is acted upon by a force or pressure, fluid pressure within the chemical chamber may cause a portion of the chemical chamber at the distal end to rupture and form an opening through which the chemical agent may exit the chemical chamber. The rupture and subsequent opening in the chemical chamber may cause the chemical chamber to come into fluid communication with one or more therapeutic elements of the catheter. Further force or pressure on the chemical chamber may collapse the chemical chamber, increasing fluid pressure in the chemical chamber and urging the predetermined volume of chemical agent through the therapeutic elements and into patient tissue.

By using any of the configurations described herein, the catheter may enable a clinician to more precisely and reliably deliver a predetermined dosage of chemical agent to a treatment site. This may improve repeatability of a chemical neuromodulation technique, may reduce a volume of chemical agent contained by the catheter and introduced to the treatment site, improve a likelihood of success of the chemical neuromodulation procedure, and/or reduce introduction of excess liquid into tissue at the treatment site.

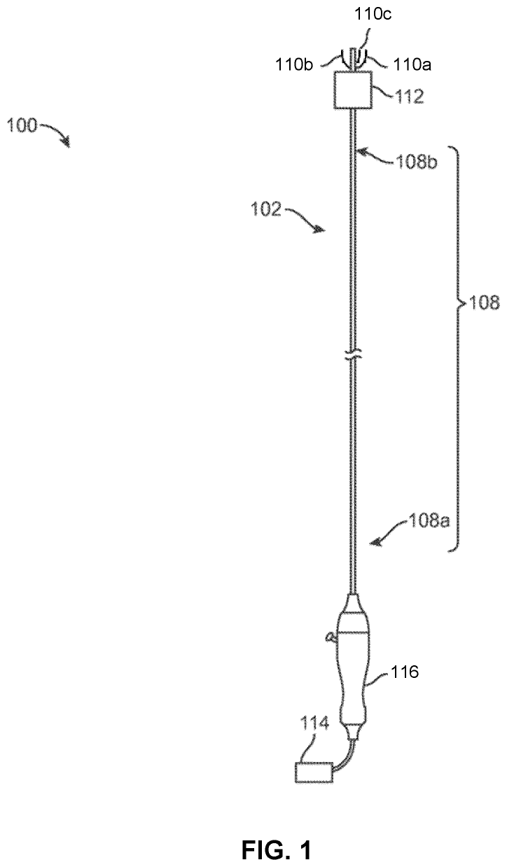

FIG. 1 is a partially schematic illustration of an example neuromodulation system 100 configured in accordance with some examples of the present disclosure. Neuromodulation system 100 includes a neuromodulation catheter 102 and an actuator 114 (shown schematically in FIG. 1 attached to an elongate shaft 108 via a handle 116). Neuromodulation catheter 102 includes an elongate shaft (also referred to as an elongate body) 108 having a proximal portion 108a and a distal portion 108b. Neuromodulation catheter 102 may further include a handle 116 operably connected to shaft 108 via proximal portion 108a and a therapeutic assembly 112 (shown schematically in FIG. 1) that is part of or attached to or carried by distal portion 108b. Shaft 108 is configured to locate the therapeutic assembly 112 at a treatment location within or otherwise proximate to a body lumen (e.g., a blood vessel, a duct, an airway, or another naturally occurring lumen within the human body). In some examples, shaft 108 is configured to locate therapeutic assembly 112 at an intraluminal (e.g., intravascular) treatment location. Therapeutic assembly 112 may be configured to provide or support a neuromodulation treatment at the treatment location.

Therapeutic assembly 112 also includes a plurality of therapeutic elements 110a-110c (“therapeutic elements 110”). Therapeutic elements 110 may be positioned around (e.g., distributed around) a perimeter (e.g., a circumference) of distal portion 108b. For example, therapeutic assembly 112 may include three therapeutic elements 110 distributed evenly around a perimeter of distal portion 108b (e.g., at 120-degree intervals).

Although distal portion 108a is shown in FIG. 1 as including three therapeutic elements 110 positioned around a circumference of distal portion 108b at a single longitudinal position along elongated shaft 108, in other examples, distal portion 108b may include any number of therapeutic elements 110. For example, distal portion 108b may include two, three, four, or more therapeutic elements 110 positioned around a perimeter of distal portion 108a at a single longitudinal position. As another example, distal portion 108b may include positioned two, three, four, or more therapeutic elements 110 positioned around a perimeter of distal portion 108a at each of multiple longitudinal positions along distal portion 108b. In examples in which distal portion 108b includes therapeutic elements 110 positioned at different longitudinal positions, each longitudinal position may include one or more therapeutic element 110, and each longitudinal position may include the same number of therapeutic elements 110, or one longitudinal position may include a different number of therapeutic elements 110 than one or more other longitudinal positions.

Elongated shaft 108 may have any suitable outer diameter, and the diameter can be constant along the length of elongated shaft 108 or may vary along the length of elongated shaft 108. In some examples, elongated shaft 108 can be 2, 3, 4, 5, 6, or 7 French or another suitable size.

In examples in which neuromodulation catheter 102 is configured to deliver a chemical agent, therapeutic elements 110 may include needles configured to be deployed to extend radially from distal portion 108a and at least partially pierce a wall of the lumen (e.g., vessel) in which distal portion 108a is positioned. The needles may extend to and/or through the intima, media, and/or adventitia of the wall and be configured to deliver the chemical agent to the adventitia and/or peri-adventitia, in which renal nerves are located. By having therapeutic elements 110 located around a perimeter of distal portion 108a, neuromodulation catheter 102 may be used to deliver the chemical agent around a perimeter of the lumen in which distal portion 108a is positioned. Again, while a perimeter or circumference of the lumen is generally referred to herein, the lumen may not be perfectly circular in cross-section and may have any suitable geometry in cross-section.

Intraluminal delivery of neuromodulation catheter 102 may include percutaneously inserting a guidewire (not shown) into a body lumen of a patient and moving shaft 108 and therapeutic assembly 112 along the guide wire until therapeutic assembly 112 reaches a suitable treatment location. Alternatively, neuromodulation catheter 102 may be a steerable or non-steerable device configured for use without a guidewire. Additionally, or alternatively, neuromodulation catheter 102 may be configured for use with another type of guide member, such as a guide catheter or a sheath (not shown), alone, or in addition to a guidewire.

Actuator 114 may be configured to a transmit a force or pressure to a chemical chamber of therapeutic assembly 112 (e.g., upon a clinician exerting a force on actuator 114). In some examples, a clinician may apply a force at a proximal end of catheter 102 (via actuator 114) along a longitudinal axis of catheter 102 that induces a pressure on or within the chemical chamber. In some examples, actuator 114 may include a syringe defining a lumen, where the lumen of the syringe is in fluid communication with a lumen defined by elongate body 108. When the syringe is depressed at actuator 114, the syringe applies a pressure to the interior of the syringe lumen that is equally transmitted through the lumen of the elongate body 108 and the chemical chamber of therapeutic assembly 112. The pressure may apply force to the chemical chamber transverse to a longitudinal axis of catheter 102. In some examples, actuator 114 may include a connection port for attaching a pressurized air line (not shown), a valve for allowing pressurized air from the air line into the lumen defined by elongate body 108, and a valve for releasing pressure from the lumen defined by elongate body 108. In some examples, actuator 114 may include a proximal end of a push member that travels through the lumen defined by elongate body 108 to a compression element next to the chemical chamber in the therapeutic assembly 112. A clinician may manipulate the proximal end of the push member to advance the compression element distally or retract the compression element proximally. The clinician may advance the compression element such that it pushes against the chemical chamber of therapeutic assembly 112.

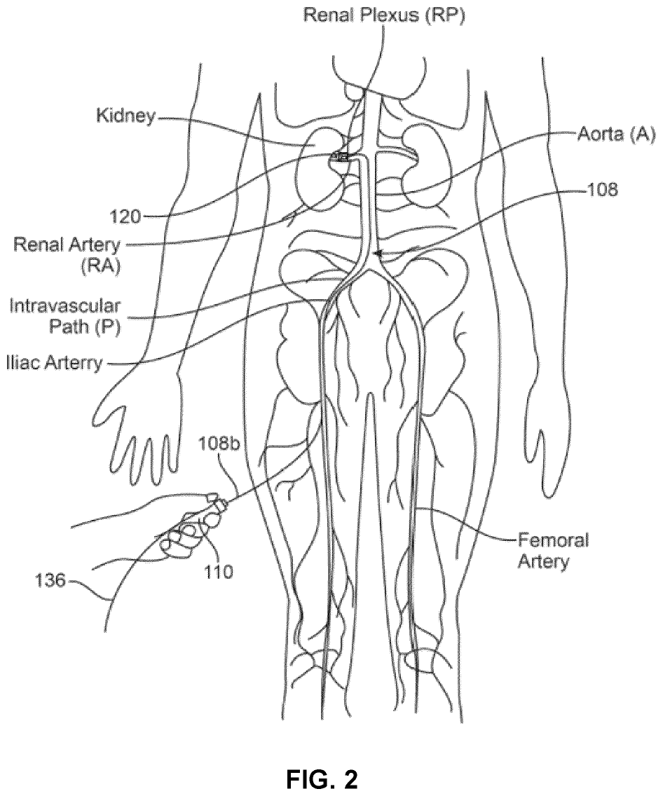

FIG. 2 (with additional reference to FIG. 1) illustrates gaining access to renal nerves of an example patient in accordance with some examples of the present technology. Neuromodulation catheter 102 provides access to the renal plexus RP through an intravascular path P, such as a percutaneous access site in the femoral (illustrated), brachial, radial, or axillary artery to a targeted treatment site within a respective renal artery RA. By manipulating proximal portion 108a of elongated shaft 108 from outside the intravascular path P, a clinician may advance at least distal portion 108b of elongated shaft 108 through the sometimes tortuous intravascular path P and remotely manipulate distal portion 108b (FIG. 1) of elongated shaft 108.

In the example illustrated in FIG. 2, therapeutic elements 110 are delivered intravascularly to the treatment site using a guidewire 136 in an OTW technique. Neuromodulation system 120 may define a passageway for receiving guidewire 136 for delivery of the neuromodulation catheter 102 using either an OTW or a RX technique. At the treatment site, guidewire 136 can be at least partially withdrawn or removed, and therapeutic elements 110 can transform or otherwise be moved to a deployed arrangement for delivering a chemical agent. In other examples, therapeutic elements 110 may be delivered to the treatment site within a different guide device, such as a guide sheath (not shown), with or without using guidewire 136. In examples in which the system includes a guide sheath, when therapeutic elements 110 are at the target site, the guide sheath may be at least partially withdrawn or retracted and therapeutic elements 110 may be transformed into the deployed arrangement. For example, at least a portion of therapeutic elements 110 may be self-expandable such that they expand to the deployed arrangement upon being released from the guide sheath. In still other examples, elongated shaft 108 may be steerable itself such that therapeutic elements 110 may be delivered to the treatment site without the aid of guidewire 136 and/or a guide sheath.

Imaging device 104 (FIG. 1) may enable image guidance, e.g., computed tomography (CT), fluoroscopy, intravascular ultrasound (IVUS), optical coherence tomography (OCT), intracardiac echocardiography (ICE), or another suitable guidance modality, or combinations thereof, to be used to aid the clinician's positioning and manipulation of distal portion 108b and therapeutic elements 110. For example, a fluoroscopy system (e.g., including a flat-panel detector, x-ray, or c-arm) can be rotated to accurately visualize and identify the target treatment site. In other examples, the target treatment site can be determined using IVUS, OCT, and/or other suitable image mapping modalities that can correlate the target treatment site with an identifiable anatomical structure (e.g., a spinal feature) and/or a radiopaque ruler (e.g., positioned under or on the patient) before delivering therapeutic elements 110. Further, in some examples, image guidance components (e.g., IVUS, OCT) may be integrated with neuromodulation catheter 102 and/or run in parallel with neuromodulation catheter 102 to provide image guidance during positioning of therapeutic elements 110. For example, image guidance components (e.g., IVUS or OCT) can be coupled to therapeutic elements 110 to provide three-dimensional images of the vasculature proximate the target site to facilitate positioning or deploying therapeutic elements 110 within the target renal blood vessel.

Renal neuromodulation is the partial or complete incapacitation or other effective disruption of nerves of the kidneys (e.g., nerves terminating in the kidneys or in structures closely associated with the kidneys). In particular, renal neuromodulation can include inhibiting, reducing, and/or blocking neural communication along neural fibers (e.g., efferent and/or afferent neural fibers) of the kidneys. Such incapacitation can be long-term (e.g., permanent or for periods of months, years, or decades) or short-term (e.g., for periods of minutes, hours, days, or weeks). Renal neuromodulation is expected to contribute to the systemic reduction of sympathetic tone or drive and/or to benefit at least some specific organs and/or other bodily structures innervated by sympathetic nerves. Accordingly, renal neuromodulation is expected to be useful in treating clinical conditions associated with systemic sympathetic overactivity or hyperactivity, particularly conditions associated with central sympathetic overstimulation. For example, renal neuromodulation is expected to efficaciously treat hypertension, heart failure, acute myocardial infarction, metabolic syndrome, insulin resistance, diabetes, left ventricular hypertrophy, chronic and end stage renal disease, inappropriate fluid retention in heart failure, cardio-renal syndrome, polycystic kidney disease, polycystic ovary syndrome, osteoporosis, erectile dysfunction, and sudden death, among other conditions.

Renal neuromodulation can be electrically induced, thermally induced, chemically-induced, or induced in another suitable manner or combination of manners at one or more suitable target sites during a treatment procedure. The target site can be within or otherwise proximate to a renal lumen (e.g., a renal artery, a ureter, a renal pelvis, a major renal calyx, a minor renal calyx, or another suitable structure), and the treated tissue can include tissue at least proximate to a wall of the renal lumen. For example, with regard to a renal artery, a treatment procedure can include modulating nerves in the renal plexus, which lay intimately within or adjacent to the adventitia of the renal artery.

As described above, some patients may have multiple arteries extending from the aorta (A) to a single kidney (i.e., a right kidney or a left kidney). One of these multiple arteries may be referred to as a main renal artery, while the other(s) of these multiple arteries may be referred to as accessory renal arteries. In patients that have multiple arteries extending from the aorta (A) to a single kidney, some renal nerves may travel along or approach a main renal artery and some nerves may travel along or approach the accessory renal artery or arteries. As such, a treatment procedure may include treating nerves along or approaching the main renal artery and the accessory renal artery or arteries.

Treatment procedures that treat at multiple target sites may require repositioning of neuromodulation catheter 102, which may increase the procedure time, complexity, and cost. Thus, combining treatments at multiple target sites into a single treatment step may save time, simplify the procedure, and reduce cost to the patient. As described above, neuromodulation catheter 102 is configured to enable treatment of nerves along or approaching a first renal vessel (such as a main renal artery) and nerves along or approaching a second renal vessel (such as an accessory renal artery) in a single treatment step.

The following discussion provides further details regarding patient anatomy and physiology as it may relate to renal denervation therapy. This section is intended to supplement and expand upon the previous discussion regarding the relevant anatomy and physiology, and to provide additional context regarding the disclosed technology and the therapeutic benefits associated with renal denervation. For example, several properties of the renal vasculature may inform the design of treatment devices and associated methods for achieving renal neuromodulation via intravascular access and impose specific design requirements for such devices. Specific design requirements may include accessing the renal artery, positioning therapeutic elements 110 within the renal artery and relative to other physiological structures (such as an accessory renal artery), delivering the chemical agent to targeted tissue, and/or effectively modulating the renal nerves with the therapy delivery device.

As noted previously, the sympathetic nervous system (SNS) is a branch of the autonomic nervous system along with the enteric nervous system and parasympathetic nervous system. It is always active at a basal level (called sympathetic tone) and becomes more active during times of stress. Like other parts of the nervous system, the sympathetic nervous system operates through a series of interconnected neurons. Sympathetic neurons are frequently considered part of the peripheral nervous system (PNS), although many lie within the central nervous system (CNS). Sympathetic neurons of the spinal cord (which is part of the CNS) communicate with peripheral sympathetic neurons via a series of sympathetic ganglia. Within the ganglia, spinal cord sympathetic neurons join peripheral sympathetic neurons through synapses. Spinal cord sympathetic neurons are therefore called presynaptic (or preganglionic) neurons, while peripheral sympathetic neurons are called postsynaptic (or postganglionic) neurons.

At synapses within the sympathetic ganglia, preganglionic sympathetic neurons release acetylcholine, a chemical messenger that binds and activates nicotinic acetylcholine receptors on postganglionic neurons. In response to this stimulus, postganglionic neurons principally release noradrenaline (norepinephrine). Prolonged activation may elicit the release of adrenaline from the adrenal medulla.

Once released, norepinephrine and epinephrine bind adrenergic receptors on peripheral tissues. Binding to adrenergic receptors causes a neuronal and hormonal response. The physiologic manifestations include pupil dilation, increased heart rate, occasional vomiting, and increased blood pressure. Increased sweating is also seen due to binding of cholinergic receptors of the sweat glands.

The sympathetic nervous system is responsible for up- and down-regulating many homeostatic mechanisms in living organisms. Fibers from the SNS innervate tissues in almost every organ system, providing at least some regulatory function to physiological features as diverse as pupil diameter, gut motility, and urinary output. This response is also known as sympatho-adrenal response of the body, as the preganglionic sympathetic fibers that end in the adrenal medulla (but also all other sympathetic fibers) secrete acetylcholine, which activates the secretion of adrenaline (epinephrine) and to a lesser extent noradrenaline (norepinephrine). Therefore, this response that acts primarily on the cardiovascular system is mediated directly via impulses transmitted through the sympathetic nervous system and indirectly via catecholamines secreted from the adrenal medulla.

Science typically looks at the SNS as an automatic regulation system, that is, one that operates without the intervention of conscious thought. Some evolutionary theorists suggest that the sympathetic nervous system operated in early organisms to maintain survival as the sympathetic nervous system is responsible for priming the body for action. One example of this priming is in the moments before waking, in which sympathetic outflow spontaneously increases in preparation for action.

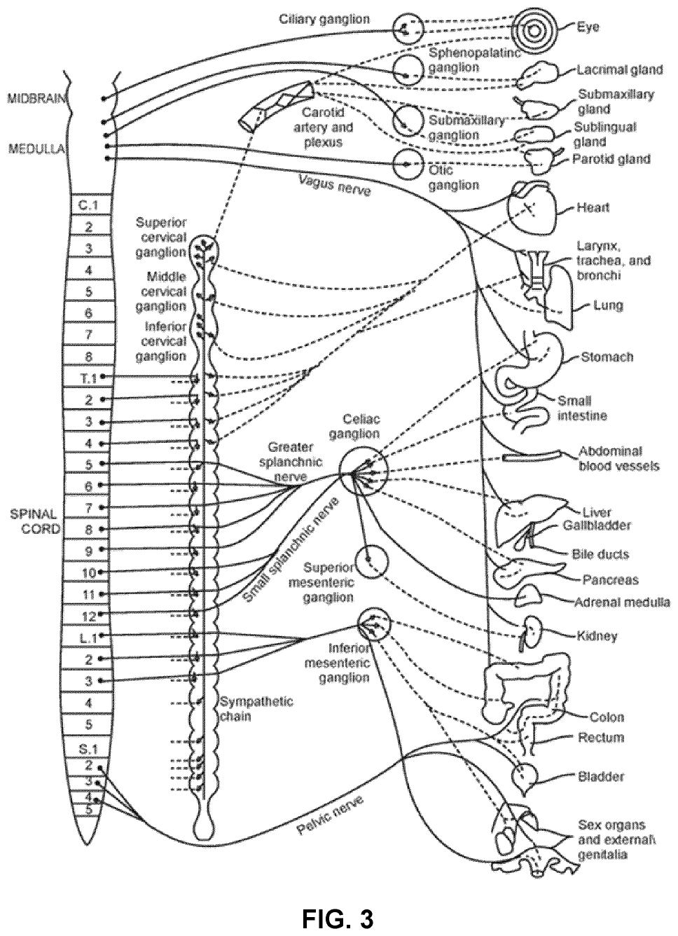

As shown in FIG. 3, the SNS provides a network of nerves that allows the brain to communicate with the body. Sympathetic nerves originate inside the vertebral column, e.g., toward the middle of the spinal cord in the intermediolateral cell column (or lateral horn), beginning at the first thoracic segment of the spinal cord and are thought to extend to the second or third lumbar segments. Because SNS cells begin in the thoracic and lumbar regions of the spinal cord, the SNS is said to have a thoracolumbar outflow. Axons of these nerves leave the spinal cord through the anterior rootlet/root. They pass near the spinal (sensory) ganglion, where they enter the anterior rami of the spinal nerves. However, unlike somatic innervation, they quickly separate out through white rami connectors which connect to either the paravertebral (which lie near the vertebral column) or prevertebral (which lie near the aortic bifurcation) ganglia extending alongside the spinal column.

In order to reach the target organs and glands, the axons should travel long distances in the body, and, to accomplish this, many axons relay their message to a second cell through synaptic transmission. The ends of the axons link across a space, the synapse, to the dendrites of the second cell. The first cell (the presynaptic cell) sends a neurotransmitter across the synaptic cleft where it activates the second cell (the postsynaptic cell). The message is then carried to the final destination.

In the SNS and other components of the peripheral nervous system, these synapses are made at sites called ganglia, discussed above. The cell that sends its fiber to the ganglion is called a preganglionic cell, while the cell whose fiber leaves the ganglion is called a postganglionic cell. As mentioned previously, the preganglionic cells of the SNS are located between the first thoracic (T1) segment and third lumbar (L3) segments of the spinal cord. Postganglionic cells have their cell bodies in the ganglia and send their axons to target organs or glands.

The ganglia include not just the sympathetic trunks but also the cervical ganglia (superior, middle, and inferior), which sends sympathetic nerve fibers to the head and thorax organs, and the celiac and mesenteric ganglia (which send sympathetic fibers to the gut).

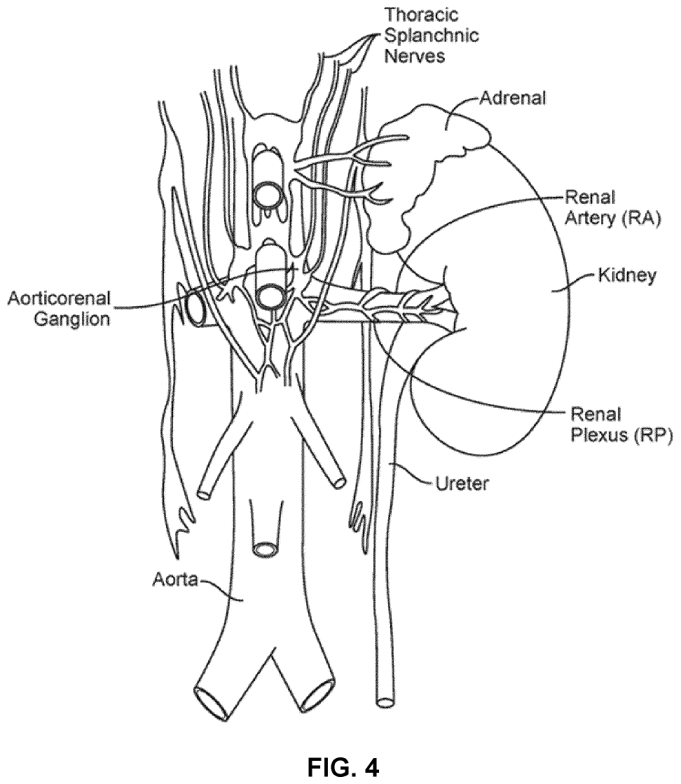

As FIG. 4 shows, the kidney is innervated by the renal plexus (RP), which is intimately associated with the renal artery. The renal plexus (RP) is an autonomic plexus that surrounds the renal artery and is embedded within the adventitia of the renal artery. The renal plexus (RP) extends along the renal artery until it arrives at the substance of the kidney. Fibers contributing to the renal plexus (RP) arise from the celiac ganglion, the superior mesenteric ganglion, the aorticorenal ganglion and the aortic plexus. The renal plexus (RP), also referred to as the renal nerve, is predominantly comprised of sympathetic components. There is no (or at least very minimal) parasympathetic innervation of the kidney.

Preganglionic neuronal cell bodies are located in the intermediolateral cell column of the spinal cord. Preganglionic axons pass through the paravertebral ganglia (they do not synapse) to become the lesser splanchnic nerve, the least splanchnic nerve, the first lumbar splanchnic nerve, the second lumbar splanchnic nerve, and travel to the celiac ganglion, the superior mesenteric ganglion, and the aorticorenal ganglion. Postganglionic neuronal cell bodies exit the celiac ganglion, the superior mesenteric ganglion, and the aorticorenal ganglion to the renal plexus (RP) and are distributed to the renal vasculature.

Messages travel through the SNS in a bidirectional flow. Efferent messages may trigger changes in different parts of the body simultaneously. For example, the sympathetic nervous system may accelerate heart rate; widen bronchial passages; decrease motility (movement) of the large intestine; constrict blood vessels; increase peristalsis in the esophagus; cause pupil dilation, piloerection (goose bumps) and perspiration (sweating); or raise blood pressure. Afferent messages carry signals from various organs and sensory receptors in the body to other organs and, particularly, the brain.

Hypertension, heart failure, and chronic kidney disease are a few of many disease states that result from chronic activation of the SNS, especially the renal sympathetic nervous system. Chronic activation of the SNS is a maladaptive response that drives the progression of these disease states. Pharmaceutical management of the renin-angiotensin-aldosterone system (RAAS) has been a longstanding, but somewhat ineffective, approach for reducing over-activity of the SNS.

As mentioned above, the renal sympathetic nervous system has been identified as a major contributor to the complex pathophysiology of hypertension, states of volume overload (such as heart failure), and progressive renal disease, both experimentally and in humans. Studies employing radiotracer dilution methodology to measure overflow of norepinephrine from the kidneys to plasma revealed increased renal norepinephrine (NE) spillover rates in patients with essential hypertension, particularly so in young hypertensive subjects, which in concert with increased NE spillover from the heart, is consistent with the hemodynamic profile typically seen in early hypertension and characterized by an increased heart rate, cardiac output, and renovascular resistance. It is now known that essential hypertension is commonly neurogenic, often accompanied by pronounced sympathetic nervous system overactivity.

Activation of cardiorenal sympathetic nerve activity is even more pronounced in heart failure, as demonstrated by an exaggerated increase of NE overflow from the heart and the kidneys to plasma in this patient group. In line with this notion is the recent demonstration of a strong negative predictive value of renal sympathetic activation on all-cause mortality and heart transplantation in patients with congestive heart failure, which is independent of overall sympathetic activity, glomerular filtration rate, and left ventricular ejection fraction. These findings support the notion that treatment regimens that are designed to reduce renal sympathetic stimulation have the potential to improve survival in patients with heart failure.

Both chronic and end stage renal disease in some patients are characterized by heightened sympathetic nervous activation. In patients with end stage renal disease, plasma levels of norepinephrine above the median have been demonstrated to be predictive for both all-cause death and death from cardiovascular disease. This can also be true for patients suffering from diabetic or contrast nephropathy. There is compelling evidence suggesting that sensory afferent signals originating from the diseased kidneys are major contributors to initiating and sustaining elevated central sympathetic outflow in this patient group; this facilitates the occurrence of the well-known adverse consequences of chronic sympathetic over activity, such as hypertension, left ventricular hypertrophy, ventricular arrhythmias, sudden cardiac death, insulin resistance, diabetes, and metabolic syndrome.

Sympathetic nerves to the kidneys terminate in the blood vessels, the juxtaglomerular apparatus, and the renal tubules. Stimulation of the renal sympathetic nerves causes increased renin release, increased sodium (Na) reabsorption, and a reduction of renal blood flow. These components of the neural regulation of renal function are considerably stimulated in disease states characterized by heightened sympathetic tone and clearly contribute to the rise in blood pressure in hypertensive patients. The reduction of renal blood flow and glomerular filtration rate as a result of renal sympathetic efferent stimulation may be a cornerstone of the loss of renal function in cardio-renal syndrome, which is renal dysfunction as a progressive complication of chronic heart failure, with a clinical course that typically fluctuates with the patient's clinical status and treatment. Pharmacologic strategies to thwart the consequences of renal efferent sympathetic stimulation include centrally acting sympatholytic drugs, beta blockers (intended to reduce renin release), angiotensin converting enzyme inhibitors and receptor blockers (intended to block the action of angiotensin II and aldosterone activation consequent to renin release), and diuretics (intended to counter the renal sympathetic mediated sodium and water retention). However, the current pharmacologic strategies can have significant limitations including limited efficacy, compliance issues, side effects and others.

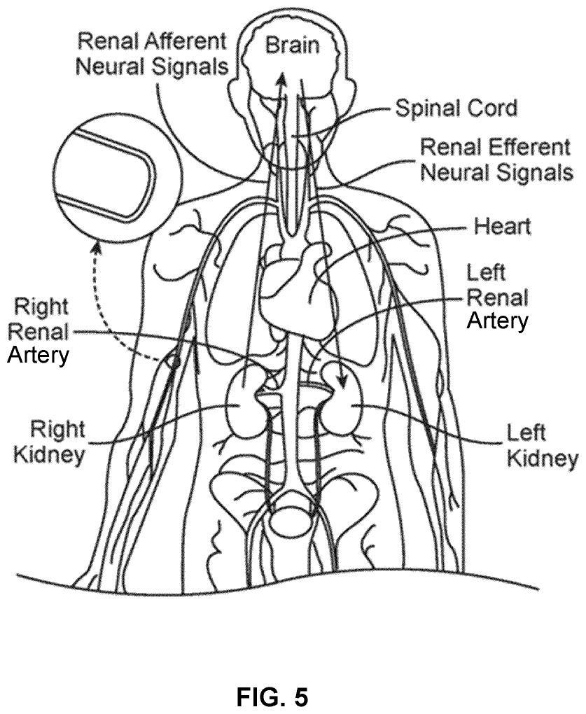

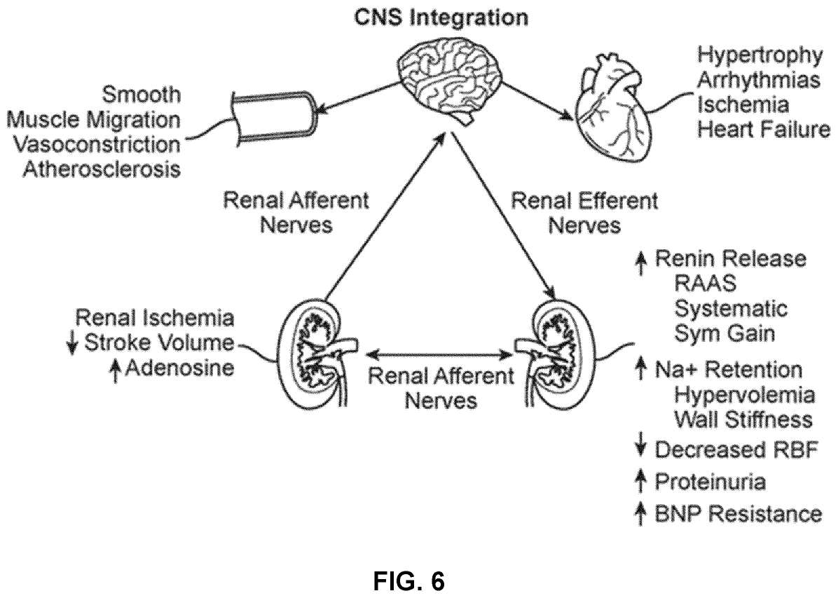

The kidneys communicate with integral structures in the central nervous system via renal sensory afferent nerves. Several forms of “renal injury” may induce activation of sensory afferent signals. For example, renal ischemia, reduction in stroke volume or renal blood flow, or an abundance of adenosine enzyme may trigger activation of afferent neural communication. As shown in FIGS. 5 and 6, this afferent communication might be from the kidney to the brain or might be from one kidney to the other kidney (via the central nervous system). These afferent signals are centrally integrated and may result in increased sympathetic outflow. This sympathetic drive is directed towards the kidneys, thereby activating the RAAS and inducing increased renin secretion, sodium retention, volume retention, and vasoconstriction. Central sympathetic over activity also impacts other organs and bodily structures innervated by sympathetic nerves such as the heart and the peripheral vasculature, resulting in the described adverse effects of sympathetic activation, several aspects of which also contribute to the rise in blood pressure.

The physiology therefore suggests that (i) modulation of tissue with efferent sympathetic nerves will reduce inappropriate renin release, salt retention, and reduction of renal blood flow, and that (ii) modulation of tissue with afferent sensory nerves will reduce the systemic contribution to hypertension and other disease states associated with increased central sympathetic tone through its direct effect on the posterior hypothalamus as well as the contralateral kidney. In addition to the central hypotensive effects of afferent renal denervation, a desirable reduction of central sympathetic outflow to various other sympathetically innervated organs such as the heart and the vasculature is anticipated.

As provided above, renal denervation is likely to be valuable in the treatment of several clinical conditions characterized by increased overall and particularly renal sympathetic activity such as hypertension, metabolic syndrome, insulin resistance, diabetes, left ventricular hypertrophy, chronic end stage renal disease, inappropriate fluid retention in heart failure, cardio-renal syndrome, and sudden death. Since the reduction of afferent neural signals contributes to the systemic reduction of sympathetic tone/drive, renal denervation might also be useful in treating other conditions associated with systemic sympathetic hyperactivity. Accordingly, renal denervation may also benefit other organs and bodily structures innervated by sympathetic nerves, including those identified in FIG. 5. For example, as previously discussed, a reduction in central sympathetic drive may reduce the insulin resistance that afflicts people with metabolic syndrome and Type II diabetics. Additionally, patients with osteoporosis may also be sympathetically activated and might also benefit from the down regulation of sympathetic drive that accompanies renal denervation.

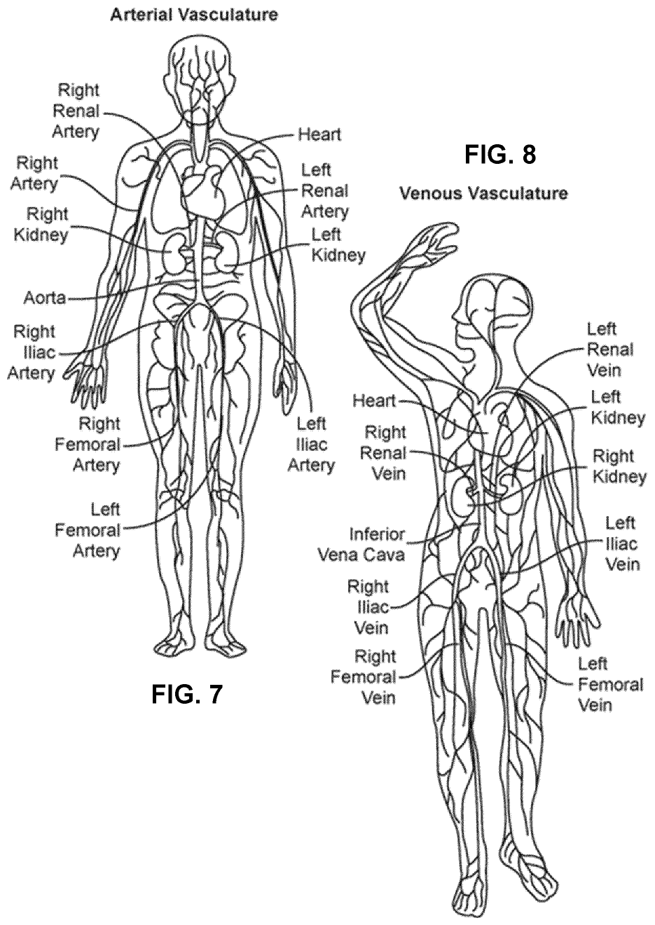

In accordance with the present technology, neuromodulation of a left and/or right renal plexus (RP), which is intimately associated with a left and/or right renal artery, may be achieved through intravascular access. As FIG. 7 shows, blood moved by contractions of the heart is conveyed from the left ventricle of the heart by the aorta. The aorta descends through the thorax and branches into the left and right renal arteries. Below the renal arteries, the aorta bifurcates at the left and right iliac arteries. The left and right iliac arteries descend, respectively, through the left and right legs and join the left and right femoral arteries.

As FIG. 8 shows, the blood collects in veins and returns to the heart, through the femoral veins into the iliac veins and into the inferior vena cava. The inferior vena cava branches into the left and right renal veins. Above the renal veins, the inferior vena cava ascends to convey blood into the right atrium of the heart. From the right atrium, the blood is pumped through the right ventricle into the lungs, where it is oxygenated. From the lungs, the oxygenated blood is conveyed into the left atrium. From the left atrium, the oxygenated blood is conveyed by the left ventricle back to the aorta.

As will be described in greater detail later, the femoral artery may be accessed and cannulated at the base of the femoral triangle just inferior to the midpoint of the inguinal ligament. A catheter may be inserted percutaneously into the femoral artery through this access site, passed through the iliac artery and aorta, and placed into either the left or right renal artery. This comprises an intravascular path that offers minimally invasive access to a respective renal artery and/or other renal blood vessels.

The wrist, upper arm, and shoulder region provide other locations for introduction of catheters into the arterial system. For example, catheterization of either the radial, brachial, or axillary artery may be utilized in select cases. Catheters introduced via these access points may be passed through the subclavian artery on the left side (or via the subclavian and brachiocephalic arteries on the right side), through the aortic arch, down the descending aorta and into the renal arteries using standard angiographic technique. Other access sites can also be used to access the arterial system.

Since neuromodulation of a left and/or right renal plexus (RP) may be achieved in accordance with the present technology through intravascular access, properties and characteristics of the renal vasculature may impose constraints upon and/or inform the design of apparatus, systems, and methods for achieving such renal neuromodulation. Some of these properties and characteristics may vary across the patient population and/or within a specific patient across time, as well as in response to disease states, such as hypertension, chronic kidney disease, vascular disease, end-stage renal disease, insulin resistance, diabetes, metabolic syndrome, and the like. These properties and characteristics, as explained herein, may have bearing on the efficacy of the procedure and the specific design of the intravascular device. Properties of interest may include, for example, material/mechanical, spatial, fluid dynamic/hemodynamic and/or thermodynamic properties.

As discussed previously, a catheter may be advanced percutaneously into either the left or right renal artery via a minimally invasive intravascular path. However, minimally invasive renal arterial access may be challenging, for example, because as compared to some other arteries that are routinely accessed using catheters, the renal arteries are often extremely tortuous, may be of relatively small diameter, and/or may be of relatively short length. Furthermore, renal arterial atherosclerosis is common in many patients, particularly those with cardiovascular disease. Renal arterial anatomy also may vary significantly from patient to patient, which further complicates minimally invasive access. Significant inter-patient variation may be seen, for example, in relative tortuosity, diameter, length, and/or atherosclerotic plaque burden, as well as in the take-off angle at which a renal artery branches from the aorta. Further, some patients include multiple left renal arteries and/or right renal arteries. Apparatus, systems, and methods for achieving renal neuromodulation via intravascular access should account for these and other aspects of renal arterial anatomy and its variation across the patient population when minimally invasively accessing a renal artery.

In addition to complicating renal arterial access, specifics of the renal anatomy also complicate establishment of stable contact between neuromodulatory apparatus and a luminal surface or wall of a renal artery. For example, navigation can be impeded by the tight space within a renal artery, as well as tortuosity of the artery. Furthermore, establishing consistent contact is complicated by patient movement, respiration, and/or the cardiac cycle because these factors may cause significant movement of the renal artery relative to the aorta, and the cardiac cycle may transiently distend the renal artery (i.e., cause the wall of the artery to pulse).

Even after accessing a renal artery and facilitating stable contact between neuromodulatory apparatus and a luminal surface of the artery, nerves in and around the adventitia of the artery should be safely modulated via the neuromodulatory apparatus. Effectively applying chemical treatment from within a renal artery is non-trivial given the potential clinical complications associated with such treatment. For example, the intima and media of the renal artery may be vulnerable to chemical injury. As discussed in greater detail below, the intima-media thickness separating the vessel lumen from its adventitia means that target renal nerves may be multiple millimeters distant from the luminal surface of the artery. The chemical agent should be delivered to the target renal nerves to modulate the target renal nerves without excessively adversely impacting the vessel wall to the extent that the vessel wall is potentially affected to an undesirable extent.

The neuromodulatory apparatus should also be configured to allow for adjustable positioning and repositioning of the therapeutic elements 110 (FIG. 1) within the renal artery since location of treatment may also impact clinical efficacy. Additionally, variable positioning and repositioning of the neuromodulatory apparatus may prove to be useful in circumstances where the renal artery is particularly tortuous or where there are proximal branch vessels off the renal artery main vessel, making treatment in certain locations challenging.

As noted above, an apparatus positioned within a renal artery should be configured so that therapeutic elements 110 may intimately contact the vessel wall and/or extend at least partially through the vessel wall. Renal artery vessel diameter, D_{RA}, typically is in a range of about 2-10 millimeters (mm), with most of the patient population having a D_{RA} of about 4 mm to about 8 mm and an average of about 6 mm. Renal artery vessel length, L_{RA}, between its ostium at the aorta/renal artery juncture and its distal branchings, generally is in a range of about 5-70 mm, and a significant portion of the patient population is in a range of about 20-50 mm. Since the target renal plexus is embedded within the adventitia of the renal artery, the composite Intima-Media Thickness, IMT, (i.e., the radial outward distance from the artery's luminal surface to the adventitia containing target neural structures) also is notable and generally is in a range of about 0.5-2.5 mm, with an average of about 1.5 mm. Although a certain depth of treatment is important to reach the target neural fibers, the treatment should not be too deep (e.g., >10 mm from inner wall of the renal artery) to avoid non-target tissue and anatomical structures such as anatomical structures of the digestive system or psoas muscle.

An additional property of the renal artery that may be of interest is the degree of renal motion relative to the aorta induced by respiration and/or blood flow pulsatility. A patient's kidney, which is located at the distal end of the renal artery, may move as much as 10 centimeters cranially with respiratory excursion. This may impart significant motion to the renal artery connecting the aorta and the kidney, thereby requiring from the neuromodulatory apparatus a unique balance of stiffness and flexibility to maintain contact between the energy delivery element and the vessel wall during cycles of respiration. Furthermore, the take-off angle between the renal artery and the aorta may vary significantly between patients, and also may vary dynamically within a patient, e.g., due to kidney motion. The take-off angle generally may be in a range of about 30°-135°.

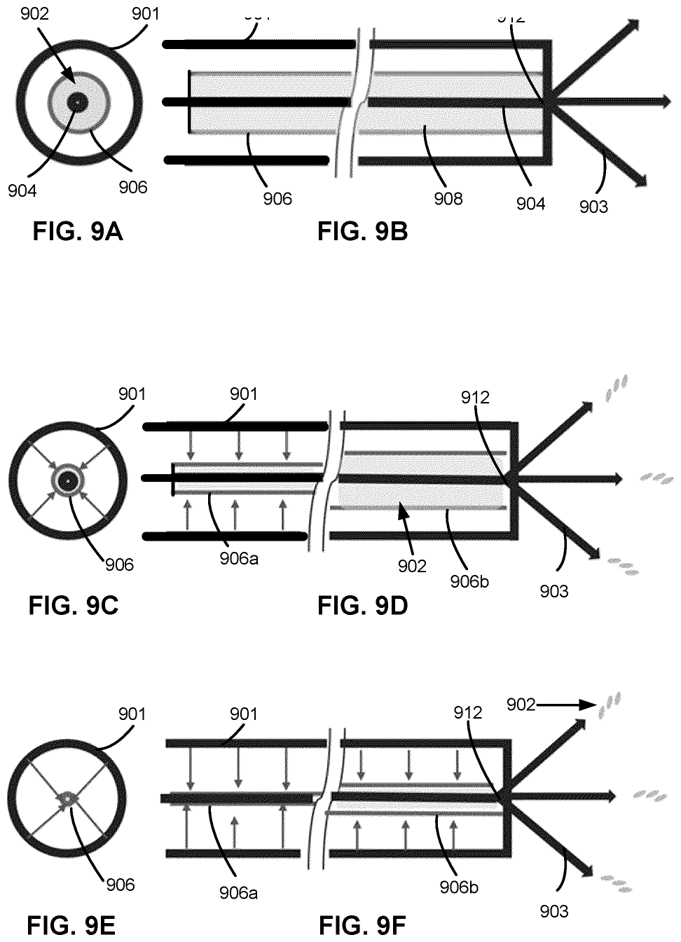

FIG. 9A is a cross-section view conceptual diagram of elongate body 901 of a catheter and an example compliant chemical chamber defined by tube 906 in an uncompressed state, in accordance with some examples of the disclosure. Elongate body 901 may be an example of elongate body 108 of catheter 102. Therapeutic elements 903 may be examples of therapeutic elements 110 of catheter 102.

In some examples, a therapeutic assembly includes a chemical chamber defined by a tube 906. Tube 906 contains a chemical agent 902 within the chemical chamber. Tube 906 is within elongate body 901 and is configured to house and supply a predetermined volume of chemical agent 902 to the treatment site (e.g., tissue) adjacent the therapeutic assembly. One or more walls of tube 906 may be compliant, such that the one or more walls deform when acted upon by a force transmitted within the distal portion of elongate body 901 (e.g., a fluid pressure within the space between walls of elongate body 901 and walls of tube 906). For example, tube 906 may be formed of a relatively flexible polymer with sufficiently thin walls to allow forces acting on tube 906 during use of the catheter to deform one or more walls of tube 906. As an example, tube 906 may include a polyurethane, a silicone, or another relatively flexible polymer formed in a relatively thin-walled tube.

FIG. 9B is a side view conceptual diagram of elongate body 108 of catheter 102 and example tube 906 in the uncompressed state of FIG. 9A, in accordance with some examples of the disclosure.

Tube 906 surrounds a therapeutic element shaft 904. Therapeutic element shaft 904 defines a lumen in fluid communication with the chemical chamber defined by tube 906 through an opening 912 in tube 906. The lumen of therapeutic element shaft 904 may also be in fluid communication with one or more lumens defined by therapeutic elements 903 when therapeutic elements 903 are deployed from elongate body 901. Before therapeutic elements 901 are deployed from elongate body 901 (e.g., into tissue of the patient at the treatment site), the lumens of therapeutic elements 903 and therapeutic element shaft 904 may not align or may otherwise be closed, preventing chemical agent 902 from exiting the chemical chamber defined by tube 906.

In some examples, opening 912 may not be defined in tube 906 until after a force is applied to tube 906. For example, tube 906 may be sealed at both a proximal and distal end, and tube 906 may contain only the predetermined volume of chemical agent. When tube 906 is acted upon by a force, fluid pressure may build within tube 906, which may cause a portion of tube 906 at the distal end to rupture to form opening 912 through which the chemical agent may exit tube 906. The rupture and subsequent opening in the chemical chamber may cause the chemical chamber to come into fluid communication with the lumens of therapeutic element shaft 904 and/or therapeutic elements 903. Further force on the chemical chamber may collapse the chemical chamber, increase fluid pressure within the chemical chamber, and urge the predetermined volume of chemical agent through the therapeutic elements and into patient tissue. In examples in which tube 906 is sealed at the proximal end of tube 906, the seal at the proximal end of tube 906 and walls of tube 906 may be sufficiently strong such that the distal end of tube 906 ruptures at a lower fluid pressure than the proximal end of tube 906 and walls of tube 906.

Tube 906 may contain a predetermined volume 908 of chemical agent 902 for treatment of patient tissue at a treatment site. As the one or more compliant walls of tube 906 are compressed (e.g., due to a force applied by a clinician at the proximal end of elongate body 901, e.g., using actuator 114), chemical agent 902 is urged through an opening 912 and out therapeutic elements 110 to the treatment site. When the one or more walls of tube 906 are fully deformed (e.g., compressed radially inwardly), predetermined volume 908 will have flowed out of tube 906 and into the treatment site (e.g., patient tissue). It should be understood that a residual amount of chemical agent 902 may remain within chemical chamber 906 and or the lumens defined by therapeutic element shaft 904 and therapeutic elements 903, and that this residual amount may be compensated for when determining the amount of chemical agent 902 to load into chemical chamber 906 before delivery. Descriptions of a predetermined amount of chemical agent flowing into patient tissue throughout this disclosure should be read with such marginal tolerances in mind.

FIG. 9C is a cross-section view conceptual diagram of elongate body 901 of the catheter and an example compliant tube 906 of FIG. 9A in a partially compressed state, in accordance with some examples of the disclosure. FIG. 9D is a side view conceptual diagram of elongate body 901 of the catheter and example tube 906 in the partially compressed state of FIG. 9C, in accordance with some examples of the disclosure. When acted on by an external force, the one or more compliant walls of tube 906 may deform (e.g., compress), and fluid pressure may build within tube 906, which may urge chemical agent 902 out of opening 912. The external force may be applied to tube 906 transverse to a longitudinal axis of the catheter. For example, the external force may be pressurized air or other pressurized fluid (e.g., water, saline, or the like) within elongated shaft 901 surrounding tube 906. The pressurized fluid exerts a pressure on walls of tube 906.

In some examples, tube 906 includes a proximal end and a distal end. The one or more walls of tube 906 include a proximal wall portion 906a and a distal wall portion 906b. In some implementations, proximal wall portion 906a may be configured to compressibly deform under a lesser force than distal wall portion 906b. For example, proximal wall portion 906a may be made from a different material than distal wall portion 906b, a different mix of materials than distal wall portion 906b or may be thinner than distal wall portion 906b. In some of these examples, a first pressure may be applied inside elongate body 901 that compressibly deforms proximal wall portion 906a and urges chemical agent 902 out of opening 912 and therapeutic elements 903 and into the treatment site.

FIG. 9E is a cross-section view conceptual diagram of elongate body 901 of the catheter and an example tube 906 of FIG. 9A in another compressed state, in accordance with some examples of the disclosure. FIG. 9F is a side view conceptual diagram of elongate body 901 of the catheter and example compliant chemical chamber in the compressed state of FIG. 9E, in accordance with some examples of the disclosure.

A second pressure greater than or equal to the first pressure may be applied inside elongate body 901 that compressibly deforms distal wall portion 906b and urges the remainder of chemical agent 902 through opening 912 and therapeutic elements 903 and into the treatment site. When both proximal wall portion 906a and distal wall portion 906b are fully compressed, predetermined volume 908 will have substantially flowed out of chemical chamber 906 and into the treatment site (e.g., patient tissue).

In some examples, tube 906 may include a proximal end and a distal end, and a length between the proximal and distal ends. The one or more walls of tube 906 may be configured to variably deform along the length, such that the force necessary to compressibly deform the one or more walls of tube 906 becomes progressively larger along the length from the proximal end to the distal end. In some examples, the length and cross-sectional area of tube 906 may define a volume equal to the predetermine volume or dose of chemical agent desired for treatment at the treatment site. A progressively larger pressure may be applied inside elongate body 901 so that chemical agent 902 is squeezed out of chemical chamber 906 from the proximal end to the distal end, ensuring that the entire predetermined volume 908 is urged out of chemical chamber 906 and into the treatment site.

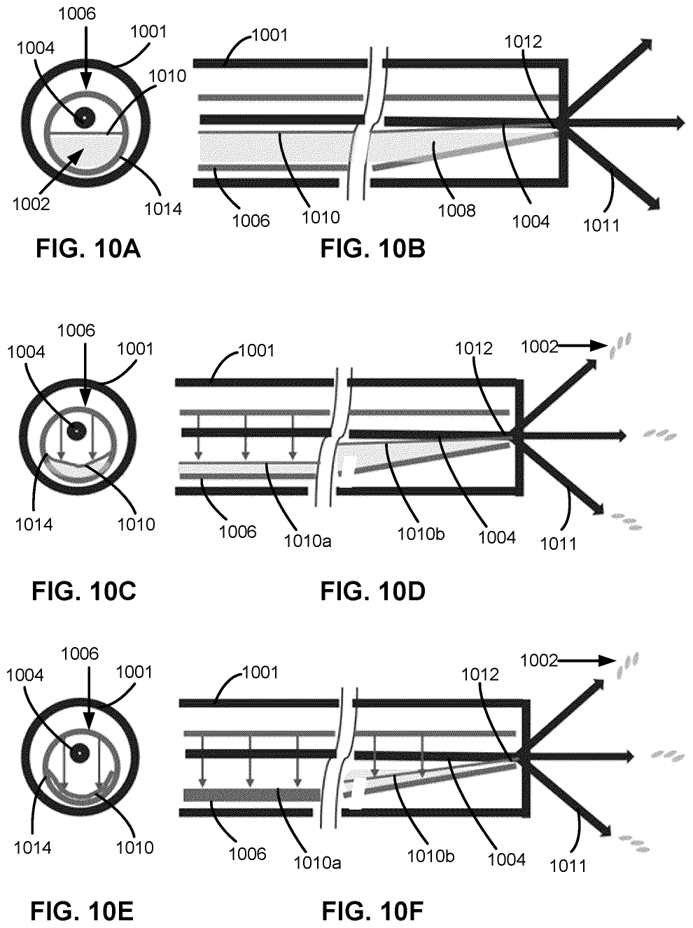

In some implementations, rather than the chemical chamber including a substantially uniform diameter in the filled state, the chemical chamber may include a variable diameter, which may be selected to affect a release pressure and/or release rate of the chemical agent from the chemical chamber to the treatment site. For example, FIG. 10A is a cross-section view conceptual diagram of an example elongate body 1001 of a catheter and another example compliant chemical chamber in an uncompressed state, in accordance with some examples of the disclosure. FIG. 10B is a side view conceptual diagram of elongate body 1001 of the catheter and example compliant chemical chamber in the uncompressed state of FIG. 10A, in accordance with some examples of the disclosure. Elongate body 1001 is an example of elongate body 108 of catheter 102.

In some examples, chemical chamber 1006 is defined by an outer tube 1014 within elongate body 1001. A separation film 1010 within tube 1014 may divide chemical chamber 1006 into a first chamber portion and a second chamber portion. Separation film 1010 may be compliant such that it deforms when acted upon by a force exerted by a force transmitted within the distal portion of the elongate body. For example, separation film 1010 may be formed of a relatively flexible polymer with sufficiently thin walls to allow forces acting on separation film 1010 during use of the catheter to deform separation film 1010. As an example, separation film 1010 may include a polyurethane, a silicone, or another relatively flexible polymer formed in a relatively thin-walled tube. Tube 1014 of chemical chamber 1006 may be noncompliant or at least less compliant than separation film 1010, such that tube 1014 does not deform when acted upon by the force transmitted within the distal portion of elongate body that deforms separation film 1010. In some examples, a proximal end of chemical chamber 1006 is spaced distally from the proximal end of the catheter, the distal end of a handle, or both. In some examples, the proximal end of the chemical chamber is located in the distal portion of elongate body 108 such that the entirety of chemical chamber 1106 is positioned within the distal portion of elongate body.

In some examples, the first chamber portion contains predetermined volume 1008 of chemical agent 1002, and the second chamber portion contains therapeutic element shaft 1004 as shown in FIGS. 10A-10F. Chemical agent 1002 and therapeutic element shaft 1004 may be similar to or substantially the same as chemical agent 902 and therapeutic element shaft 1004 described with reference to FIGS. 9A-9F, aside from differences described herein. In other examples, predetermined volume 1008 of chemical agent 1002 is contained in the same chamber portion as therapeutic element shaft 1004. Opening 1012 in chemical chamber 1006 is in fluid communication with a lumen defined by therapeutic element shaft 1004 and/or a lumen or lumens defined by therapeutic elements 1011. Before therapeutic elements 1011 are deployed into the treatment site, the lumen of therapeutic elements 1011 may not align with a lumen of therapeutic element shaft 1004 or with opening 1012, or may be otherwise blocked or closed, preventing chemical agent 1002 from exiting chemical chamber 1006.

In some examples, opening 1012 may not be defined in chemical chamber 1006 until after a force is applied to chemical chamber 1006. For example, chemical chamber 1006 may be sealed at both a proximal and distal end, and chemical chamber 1006 may contain only the predetermined volume of chemical agent. When chemical chamber 1006 is acted upon by a force, a portion of chemical chamber 1006 at the distal end ruptures to form opening 1012 through which chemical agent 1002 may exit chemical chamber 1006. The rupture and subsequent opening in chemical chamber 1006 may cause chemical chamber 1006 to come into fluid communication with the lumens of therapeutic element shaft 1004 and/or therapeutic elements 1011. Further force on chemical chamber 1006 may collapse chemical chamber 1006 and urge the predetermined volume of chemical agent 1002 through therapeutic elements 1011 and into patient tissue. In examples where chemical chamber 1006 is sealed at the proximal end of chemical chamber 1006, a force acting on chemical chamber 1006 may allow chemical agent 1002 to only exit tube 1014 distally.

FIG. 10C is a cross-section view conceptual diagram of elongate body 1001 of the catheter and the example compliant chemical chamber 1006 of FIG. 10A in a partially compressed state, in accordance with some examples of the disclosure. FIG. 10D is a side view conceptual diagram of elongate body 1001 of the catheter and the example compliant chemical chamber 1006 in the partially compressed state of FIG. 10C, in accordance with some examples of the disclosure.

When acted on by a force, compliant separation film 1010 of chemical chamber 1006 may deform, urging chemical agent 1002 out of opening 1012. The force may be applied to separation film 1010 transverse to a longitudinal axis of the catheter. For example, the force may be pressurized air or other pressurized fluid (e.g., water, saline, or the like) within a chamber portion of tube 1014. When predetermined volume 1008 of chemical agent 1002 is contained in the first chamber portion, the force may be pressurized air or other pressurized fluid applied within the second chamber portion. When predetermined volume 1008 of chemical agent 1002 is contained in the second chamber portion, the force may be applied within the first chamber portion. The chamber portion that does not contain chemical agent 1002 may expand to apply a force to the chamber that does contain chemical agent 1002, collapsing the chamber containing chemical agent 1002. For ease of description, the following paragraphs will refer to the chemical chamber portion and the force chamber portion.

In some examples, separation film 1010 of chemical chamber 1006 includes a proximal wall portion 1010a and a distal wall portion 1010b. Proximal wall portion 1010a may be configured to compressibly deform under a lesser force than distal wall portion 1010b. For example, proximal wall portion 1010a may be made from a different material than distal wall portion 1010b, a different mix of materials than distal wall portion 1010b or may be thinner than distal wall portion 1010b. In these examples, a first pressure is applied inside the force chamber portion of chemical chamber 1006 that deforms proximal wall portion 1010a and starts urging chemical agent 902 out of opening 1012 and therapeutic elements 1011 and into the treatment site (e.g., patient tissue).

FIG. 10E is a cross-section view conceptual diagram of elongate body 1001 of the catheter and an example compliant chemical chamber 1006 of FIG. 10A in another compressed state, in accordance with some examples of the disclosure. FIG. 10F is a side view conceptual diagram of elongate body 1001 of the catheter and example compliant chemical chamber 1006 in the compressed state of FIG. 10E, in accordance with some examples of the disclosure.

A second pressure greater than the first pressure may be applied inside the force chamber portion of chemical chamber 1006 to deform distal wall portion 1010b and urge the remainder of chemical agent 1002 through opening 1012 and therapeutic elements 1011 and into the treatment site (e.g., patient tissue). When both proximal wall portion 1006a and distal wall portion 1006b are fully compressed, predetermined volume 1008 will have substantially flowed out of chemical chamber 1006 and into the treatment site (e.g., patient tissue).

In some examples, chemical chamber 1006 may include a proximal end and a distal end, and a length between the proximal and distal ends. Separation film 1010 may be configured to variably deform along the length, such that the force necessary to deform separation film 1010 of chemical chamber 1006 becomes progressively larger along the length from the proximal end to the distal end. A progressively larger pressure may be applied inside the force chamber portion so that chemical agent 1002 is squeezed out of chemical chamber 1006 from the proximal end to the distal end, ensuring that the entire predetermined volume 1008 is urged out of chemical chamber 1006 and into the treatment site (e.g., patient tissue).

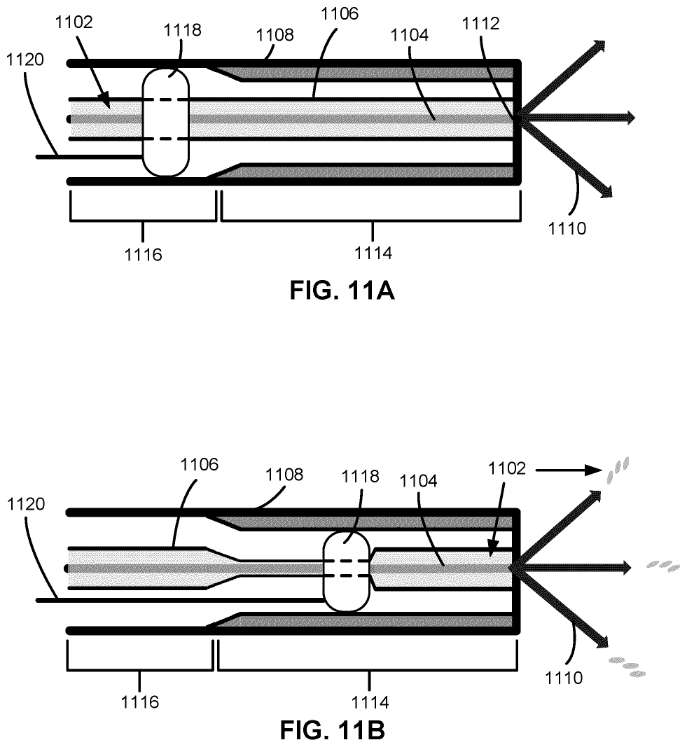

In some examples, rather than using a fluid to apply force to a compliant structure within the distal portion of the catheter, a push member may be used to apply the force. For example, FIGS. 11A and 11B are side view conceptual diagrams of an example compliant chemical chamber 1106, including a flexible ring 1118, in two different phases of compression, in accordance with some examples of the disclosure.

In some examples, the distal portion of elongate body 1108 includes a relatively large inner diameter portion 1116 and a relatively reduced inner diameter portion 1114, where relatively reduced inner diameter portion 1114 has a smaller internal diameter than relatively large inner diameter portion 1116. In the example of FIGS. 11A and 11B, relatively reduced inner diameter portion 1114 is positioned distal to relatively large inner diameter portion 1116. In some examples, relatively reduced inner diameter portion 1114 may be defined by a thicker layer of material of elongated shaft 1108, an added layer of material to the interior surface of elongated shaft 1108 in relatively reduced inner diameter portion 1114, or an area where both the outer and inner diameters of elongated shaft 1108 are reduced. Chemical chamber 1106 is filled with at least the predetermined volume of chemical agent 1102 to be dispensed from the catheter and is positioned at least within relatively reduced inner diameter portion 1114 of elongate body 1108. Opening 1112 in chemical chamber 1106 is in fluid communication with a lumen defined by therapeutic element shaft 1104 and/or a lumen or lumens defined by therapeutic elements 1110.

A push member 1120, such as a rigid rod, is attached at a distal end of push member 1120 to a compression element. In the example of FIGS. 11A and 11B, the compression element is a flexible ring 1118. Push member 1120 may be of sufficient length that a proximal end of push member 1120 is positioned within handle 116 or actuator 114 (FIG. 1). A clinician may manipulate the proximal end of push member 1120 (or actuator 114) to apply a force to the compression element through manipulation (e.g., distal advancing) of push member 1120, allowing the clinician to advance or retract flexible ring 1118 within elongated shaft 1108.

Flexible ring 1118 surrounds chemical chamber 1106 within elongate body 1108. When flexible ring 1118 is positioned within relatively large inner diameter portion 1116, flexible ring 1118 does not apply any force to the compliant walls of chemical chamber 1106. As the clinician advances push member 1120 distally, the distal end of push member 1120 pushes flexible ring 1118, and flexible ring 1118 advances distally from relatively large inner diameter portion 1116 to relatively reduced inner diameter portion 1114. Flexible ring 1118 deforms and constricts both its inner and outer diameter as it passes into relatively reduced inner diameter portion 1114, deforming the compliant wall(s) of chemical chamber 1106 and urging chemical agent 1102 through opening 1112 of chemical chamber 1106 and through the lumen defined by therapeutic element shaft 1104 and or the lumens defined by therapeutic elements 1110. Flexible ring 1118 may be made of a material that is sufficiently compliant to deform and constrict when advanced into relatively reduced inner diameter portion 1114, but also sufficiently stiff to constrict chemical chamber 1106.

Relatively reduced inner diameter portion 1114 may include a proximal end and a distal end with a length therebetween such that the volume of chemical agent 1102 in chemical chamber 1106 positioned within the length is equal to the predetermined volume of chemical agent 1102. Once flexible ring 1118 has advanced through relatively reduced inner diameter portion 1114, e.g., to a distal end of relatively reduced inner diameter portion 1114, the predetermined volume of chemical agent 1102 has passed through the opening and into the treatment site (e.g., patient tissue) through the port or needle, and no further chemical agent enters the treatment site without first distally retracting flexible ring 1118 into relatively large inner diameter portion 1116.

In some examples, chemical chamber 1106 extends through relatively reduced inner diameter portion 1114 and at least a portion of relatively large inner diameter portion 1116. In these examples, chemical chamber 1106 may contain substantially more than a predetermined volume of chemical agent 902 (i.e., more than one dose of chemical agent). A clinician may advance flexible ring 1118 all the way through relatively reduced inner diameter portion 1114 to deliver a predetermined volume of chemical agent 1102 to patient tissue at a first treatment site. The clinician may then retract flexible ring 1118 proximally into relatively large inner diameter portion 1116. After retracting flexible ring 1118, there is no longer a constrictive force on chemical chamber 1106, and more chemical agent 902 may flow into the portion of chemical chamber 1106 positioned in relatively reduced inner diameter portion 1114. In some examples, the interior of chemical chamber 1106 may be in fluid communication with a syringe or other pressurizing mechanism the clinician may use to urge more chemical agent 1102 into the portion of chemical chamber 1106 positioned in relatively reduced inner diameter portion 1114. In this way, the clinician may navigate the catheter and therapeutic assembly to a second treatment site to supply a second predetermined volume of chemical agent 1102 to the second treatment site without fully retracting the catheter or therapeutic assembly from the patient's body. The clinician may navigate the catheter and the therapeutic assembly to a second treatment site before or after retracting flexible ring 1118 into relatively large inner diameter portion 1116.

In some examples, the distal portion of elongate body 108 may not include a relatively large inner diameter portion 1116 and a relatively reduced inner diameter portion 1114 but may have a consistent inner diameter along its entire length. In some of these examples, the therapeutic assembly may include a pull wire extending the length of elongate body 1108 and attached at a distal end of the pull wire to flexible ring 1118. A proximal end of the pull wire may extend to actuator 114 or handle 116 (FIG. 1), where it may be manipulated by a clinician. A distal portion of the pull wire may be attached to flexible ring 1118 and positioned around an inner diameter of flexible ring 1118, such that when the pull wire is pulled taught, it constricts the inner diameter of flexible ring 1118 around compliant chemical chamber 1106. A clinician may apply a compression force to chemical chamber 1106 with the pull wire, and advance flexible ring 1118 distally through the distal portion of elongate body 1108 (e.g., using push member 1120) to supply patient tissue with a predetermine volume of chemical agent 1102. In some examples, a stopper may be positioned a length away from a distal end of chemical chamber 1106 such that flexible ring 1118 is prevented from advancing distally farther than necessary to deliver the predetermined volume of chemical agent 1102 when flexible ring 1118 is constricted around chemical chamber 1106 and advanced along the length.

Although ring 1118 is described with reference to FIGS. 11A and 11B as a flexible ring, in some examples ring 1118 is not flexible. For example, ring 1118 may be noncompliant, with an inner diameter similar to that depicted in FIG. 11B, where the inner diameter of ring 1118 may be slightly larger than an outer diameter of therapeutic element shaft 1104. As ring 1118 is advanced distally through elongate body 1108, ring 1118 collapses chemical chamber 1106, urging chemical agent 1102 through opening 1112 of chemical chamber 1106 and through the lumen defined by therapeutic element shaft 1104 and or the lumens defined by therapeutic elements 1110. In some examples, a proximal end of chemical chamber 1106 is sealed and located within relatively reduced inner diameter portion 1114, such that the entirety of chemical chamber 1106 is positioned within relatively reduced inner diameter portion 1114. The entirety of chemical chamber 1106 may contain a predetermined volume or dose of chemical agent 1102. As ring 1118 is advanced through relatively reduced inner diameter portion 1114 to a distal end of elongate body 1108, ring 1118 compresses the entirety of chemical chamber 1106, delivering the predetermined does of chemical agent to a treatment site.

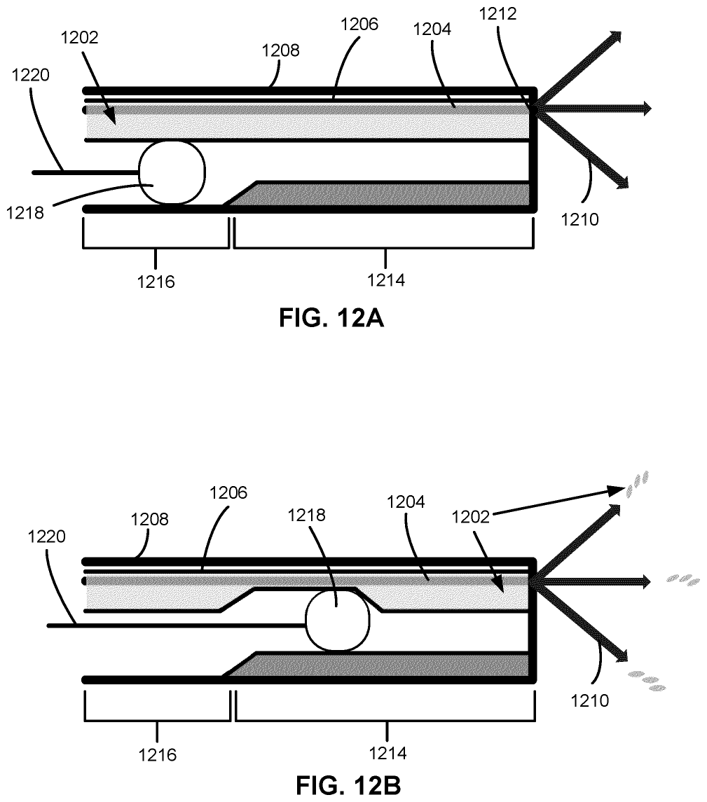

FIGS. 12A and 12B are side view conceptual diagrams of an example compliant chemical chamber 1206, including a ball 1218, in two different phases of compression, in accordance with some examples of the disclosure.

In some examples, the distal portion of elongate body 1208 includes a relatively large inner diameter portion 1216 and a relatively reduced inner diameter portion 1214, where relatively reduced inner diameter portion 1214 has a smaller internal diameter than relatively large inner diameter portion 1216. In the example of FIGS. 12A and 12B, relatively reduced inner diameter portion 1214 is positioned distal to relatively large inner diameter portion 1216. In some examples, relatively reduced inner diameter portion 1214 may be defined by a thicker layer of material of elongated shaft 1208, an added layer of material to the interior of elongated shaft 1208 in relatively reduced inner diameter portion 1214, or an area where both the outer and inner diameter of elongated shaft 108 are reduced.

Chemical chamber 1206 is filled with at least the predetermined volume of chemical agent 1202 and is positioned at least within relatively reduced inner diameter portion 1214 of elongate body 1208. Opening 1212 in chemical chamber 1206 is in fluid communication with a lumen defined by therapeutic element shaft 1204 and/or a lumen or lumens defined by therapeutic elements 1210. Although therapeutic element shaft 1204, chemical chamber 1206, and therapeutic elements 1210 are depicted in FIGS. 12A and 12B as closer to one side of elongated shaft 1208 than another, in some examples, therapeutic element shaft 1204, chemical chamber 1206, and therapeutic elements 1210 may be positioned concentrically with elongated shaft 1208, or anywhere in between.

A push member 1220 (e.g., a rigid rod) is attached at a distal end of push member 1220 to a compression element. In the example of FIGS. 12A and 12B, the compression element is a ball 1218. Push member 1220 may be of sufficient length that a proximal end of push member 1220 is positioned within handle 116 or actuator 114 (FIG. 1). A clinician may manipulate the proximal end of push member 1220 to apply a force to ball 1218 through manipulation of push member 1220, allowing the clinician to advance or retract ball 1218 within elongated shaft 1208.

Ball 1218 is positioned next to chemical chamber 1206 within elongate body 1208. When ball 1218 is positioned within relatively large inner diameter portion 1216, ball 1218 does not apply any force to the compliant walls of chemical chamber 1206. As the clinician advances push member 1220 distally, the distal end of push member 1220 pushes ball 1218, and ball 1218 advances distally from relatively large inner diameter portion 1216 to relatively reduced inner diameter portion 1214. Ball 1218 may not substantially deform as it passes into relatively reduced inner diameter portion 1214, pushing against and deforming the compliant wall of chemical chamber 1206, which in turn urges chemical agent 1202 through opening 1212 of chemical chamber 1206 and through the lumen defined by therapeutic element shaft 1204 and or the lumens defined by therapeutic elements 1210. In some examples, ball 1218 may deform slightly to maintain a smooth transition from relatively large inner diameter portion 1216 to relatively reduced inner diameter portion 1214.

Relatively reduced inner diameter portion 1214 may include a proximal end and a distal end with a length therebetween such that the volume of chemical agent 1202 in chemical chamber 1206 positioned within the length is equal to the predetermined volume of chemical agent 1202. Once ball 1218 has advanced through relatively reduced inner diameter portion 1214, the predetermined volume of chemical agent 1202 has passed through opening 1212 and therapeutic elements 1210 and into the treatment site (e.g., patient tissue), and no further chemical agent enters the treatment site without first distally retracting ball 1218 into relatively large inner diameter portion 1216.

In some examples, chemical chamber 1206 extends through relatively reduced inner diameter portion 1214 and at least a portion of relatively large inner diameter portion 1216. In these examples, chemical chamber 1206 contains substantially more than a predetermined volume of chemical agent 1202 (i.e., more than one dose of chemical agent). A clinician may advance ball 1218 of therapeutic assembly 112 all the way through relatively reduced inner diameter portion 1214 to deliver a predetermined volume of chemical agent 1202 to patient tissue at a first treatment site. The clinician may then retract ball 1218 proximally into relatively large inner diameter portion 1216. After retracting ball 1218, ball 1218 no longer exerts a constrictive force on chemical chamber 1206, and more chemical agent 1202 may flow into or be introduced into the portion of chemical chamber 1206 positioned in relatively reduced inner diameter portion 1214. In some examples, the interior of chemical chamber 1206 may be in fluid communication with a syringe or other pressurizing mechanism the clinician may use to introduce more chemical agent 1202 into the portion of chemical chamber 1206 positioned in relatively reduced inner diameter portion 1214. In this way, the clinician may navigate the catheter and therapeutic assembly to a second treatment site to supply a second predetermined volume of chemical agent 1202 without fully retracting the catheter or therapeutic assembly from the patient's body. The clinician may navigate the catheter and therapeutic assembly to a second treatment site before or after retracting ball 1218 into relatively large inner diameter portion 1216.

Although the present disclosure describes the compression element of FIGS. 12A and 12B as a “ball,” the compression element need not be spherical, and in some examples may include other shapes (e.g., cube, rectangular prism, triangular prism, hemisphere etc.). In some examples, the compression element may include a rod with a rounded end. In some examples, a distal end of push member 1220 may act as the compression element.

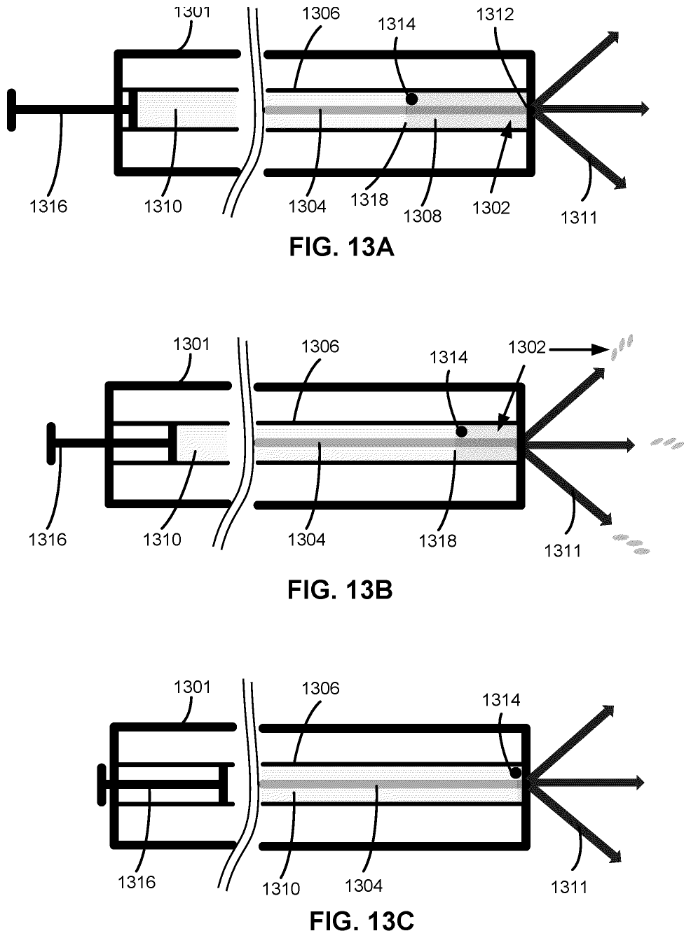

FIGS. 13A, 13B, and 13C are side view conceptual diagrams of an example syringe system in three different phases of compression, including a gel bolus of predefined volume 1308 and a plug 1314, in accordance with some examples of the disclosure. The depiction of plug 1314 in FIGS. 13A, 13B, and 13C is an example, and the size of plug 1314 may vary in other examples, so long as it is large enough to block opening 1312.

A chemical chamber 1306 may extend through elongate body 1301 and contain a predetermined volume 1308 of chemical agent 1302. Chemical chamber 1306 may not appreciably deform under the fluid pressures experienced by chemical chamber 1306 during loading, containing, and dispensing chemical agents associated with use of the catheter. In other words, in some examples, chemical chamber 1306 may be substantially rigid. In some examples, chemical chamber 1306 extends from a distal end of elongate body 1301 to an actuator 114 or handle 116 (FIG. 1) proximal to elongate body 1301 at a proximal end of chemical chamber 1306. A plug 1314 is suspended in chemical agent 1302 within chemical chamber 1306, wherein plug 1314 is larger than an opening 1312 in chemical chamber 1306. Opening 1312 is in fluid communication with a lumen defined by therapeutic element shaft 1304 and/or lumens defined by therapeutic elements 1311, allowing chemical agent 1302 to exit chemical chamber 1306 when chemical agent 1302 is acted upon by a force.

Actuator 114 (FIG. 1) may be configured to apply a force to predetermined volume 1308 of chemical agent 1302 in order to urge predetermined volume 1308 into the treatment site through opening 1312, the lumen defined by therapeutic element shaft 1304, and/or lumens defined by therapeutic elements 1311. In some examples, actuator 114 may include a syringe 1316 as shown in FIGS. 13A and 13B. In some examples, actuator 114 may include a connection port for attaching a pressurized air line (not shown). In examples where actuator 114 includes a connection port, actuator 114 may also include a valve for allowing pressurized air from the air line into the lumen defined by chemical chamber 1306, and a valve for releasing pressure from the lumen defined by chemical chamber 1306.

In some examples, chemical chamber 1306 contains predetermined volume 1308 of chemical agent 1304, as well as a filler substance 1310, wherein filler substance 1310 is proximal within chemical chamber 1306 to predetermined volume 1308 of chemical agent 1304, defining a boundary 1318 between filler substance 1310 and predetermined volume 1308 of chemical agent 1302. In some examples, filler substance 1310 may include saline. A clinician may apply a force to syringe 1316, which in turn applies pressure to filler substance 1310, which in turn applies pressure to predetermined volume 1308 of chemical agent 1304, thereby urging predetermined volume 1308 of chemical agent 1302 through opening 1312 and into the treatment site.

In some examples, plug 1314 is suspended within chemical agent 1302 or filler substance 1310 near boundary 1318. As chemical agent 1302 is urged distally out of opening 1312, plug 1314 travels distally through chemical chamber 1306. Once predetermined volume 1308 of chemical agent 1302 has exited through opening 1312, plug 1314 will be positioned at opening 1312, and will block more fluid from exiting opening 1314.

In some examples, chemical chamber 1306 is filled with substantially more than predetermined volume 1308 of chemical agent 1302 (i.e., more than one dose of chemical agent). In these examples, plug 1314 may be positioned within chemical agent 1302 a length away from opening 1312, such that as plug 1314 travels along the length towards opening 1312 when chemical agent 1302 is acted upon by a force, predetermined volume 1308 of chemical agent 1302 exits chemical chamber 1306 before plug 1314 reaches opening 1312 and prevents more chemical agent 1302 from exiting chemical chamber 1306.

In some examples, one or more of chemical agent 1302 or filler substance 1310 may be gels contained within chemical chamber 1306. Plug 1314 may be suspended in the gel near boundary 1318 to avoid floating into a different portion of chemical agent 1302 or filler substance 1310.

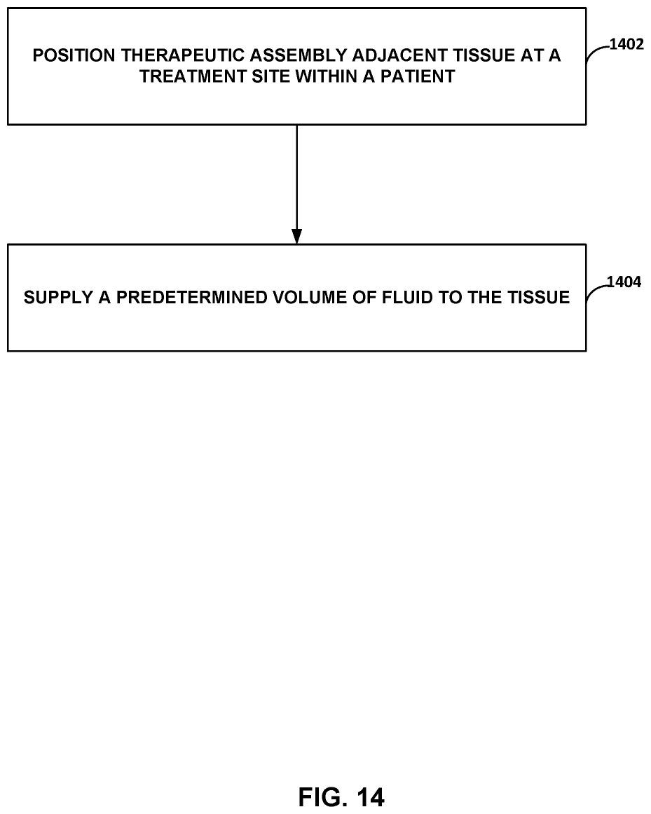

FIG. 14 is a flow diagram illustrating an example method for supplying a predetermined volume of chemical agent to a treatment site (i.e., patient tissue).

A clinician may position a therapeutic assembly of a catheter adjacent tissue at a treatment site within a patient (1402). The therapeutic assembly may be positioned on a distal portion of an elongate body of the catheter and may include a chemical chamber configured to supply a predetermined volume of chemical agent to the tissue.

The chemical agent may be useful for ablating the renal sympathetic nerves. The chemical agent may be selected to modulate activity of one or more renal nerves adjacent to the renal artery in which the catheter is positioned. For example, the chemical agent may be a neurotoxic chemical selected to chemically ablate the one or more renal nerves near the renal artery (e.g., ethanol). The catheter may include at least one port or needle through which a chemical agent is delivered. The at least one port or needle may be referred to as therapeutic elements.

Positioning of the catheter may include percutaneously inserting a guidewire into a body lumen of a patient and moving the elongate body and therapeutic assembly along the guide wire until the therapeutic assembly reaches a suitable treatment location. Alternatively, the catheter may be a steerable or non-steerable device configured for use without a guidewire. Additionally, or alternatively, the catheter may be configured for use with another type of guide member, such as a guide catheter or a sheath (not shown), alone, or in addition to a guidewire.

Once the therapeutic assembly is positioned, a clinician may supply a predetermined volume of fluid to the tissue via the chemical chamber of the therapeutic assembly (1404). Supplying a predetermined volume of fluid to the tissue may include applying a force to the chemical chamber via an actuator of the catheter.

The actuator may be configured to a transmit a force to the chemical chamber of the therapeutic assembly (e.g., upon a clinician exerting a force on the actuator). In some examples, the actuator may include a syringe defining a lumen, where the lumen of the syringe is in fluid communication with a lumen defined by the elongate body of the catheter. When the syringe is depressed at the actuator, it applies a pressure to the interior of the syringe lumen that may travel down the lumen of the elongate body to the chemical chamber of the therapeutic assembly. In some examples, the actuator may include a connection port for attaching a pressurized air line, a valve for allowing pressurized air from the air line into the lumen defined by the elongate body, and a valve for releasing pressure from the lumen defined by the elongate body. In some examples, the actuator may include a proximal end of a push member that travels through the lumen defined by the elongate body to a compression element next to the chemical chamber in the therapeutic assembly. A clinician may manipulate the proximal end of the push member to advance the compression element distally or retract the compression element proximally. The clinician may advance the compression element such that it pushes against the chemical chamber of the therapeutic assembly.

In some examples, one or more walls of the chemical chamber may be configured to deform when acted upon by the force, transmitted by the actuator within the distal portion of the elongate body. The chemical chamber may exclusively contain the predetermined volume of chemical agent (e.g., contains only the predetermined volume of chemical agent and does not include another fluid, such as saline, for example for pushing the chemical agent from the catheter). A clinician may apply a force to the chemical chamber, compressibly deforming the walls of the chemical chamber, and urging the chemical agent through an opening of the chemical chamber. The opening of the chemical chamber may be in fluid communication with a lumen defined by the port or needle through which the chemical agent exits the catheter and is supplied to the treatment site. Once the chemical chamber is fully deformed, the predetermined volume of chemical agent has passed through the opening and into the treatment site through the port or needle, and no further fluid exits the chemical chamber into the treatment site.

In some examples, a therapeutic assembly includes a chemical chamber defined by a tube containing the predetermined volume of chemical agent, where the tube is positioned within a distal portion of the elongate body of the catheter. One or more walls of the tube may be compliant, such that the one or more walls deform when acted upon by a force exerted by a force transmitted within the distal portion of elongate body. For example, the tube may be formed of a relatively flexible polymer with sufficiently thin walls to allow forces acting on the tube during use of the catheter to deform one or more walls of the tube. The tube may surround a therapeutic element shaft. The therapeutic element shaft defines a lumen in fluid communication with the chemical chamber defined by the tube through an opening in the tube. The lumen of the therapeutic element shaft may also be in fluid communication with one or more lumens defined by the therapeutic elements when the therapeutic elements are deployed from the elongate body of the catheter.

In some examples, applying the force to the chemical chamber includes pressurizing the elongate body surrounding the tube, compressing the one or more walls of the tube. As the one or more compliant walls of the tube are compressed (e.g., due to the pressure applied by a clinician at the proximal end of the elongate body, e.g., using the actuator), the chemical agent is urged through the opening and out the therapeutic elements to the treatment site. When the one or more walls of the tube are fully deformed (e.g., compressed radially inwardly), the predetermined volume will have flowed out of the tube and into the treatment site (e.g., patient tissue).

In some implementations, rather than the chemical chamber including a substantially uniform diameter in the filled state, the chemical chamber may include a variable diameter, which may be selected to affect a release pressure and/or release rate of the chemical agent from the chemical chamber to the treatment site.

In some examples, the chemical chamber is defined by a tube within the elongate body. A separation film within the tube may divide the chemical chamber into a first chamber portion and a second chamber portion. The separation film may be compliant such that it deforms when acted upon by a force exerted by a force transmitted within the first chamber portion of the therapeutic assembly (e.g., due to the pressure applied by a clinician at the proximal end of the elongate body, e.g., using the actuator). The predetermined volume of the chemical agent may be contained in the second chamber portion. When acted on by a force, the compliant separation film of the chemical chamber may deform, urging the chemical agent out of an opening in the second chamber portion in fluid communication with a lumen defined by the therapeutic elements. Applying a force to the chemical chamber may include pressurizing the first chamber portion of the therapeutic assembly, thereby deforming the separation film and urging the chemical agent through the therapeutic elements and to the treatment site.

In some examples, rather than using a fluid to apply force to a compliant structure within the distal portion of the catheter, a push member may be used to apply the force. For example, the distal portion of the elongate body may include a relatively large inner diameter portion and a relatively reduced inner diameter portion, where the relatively reduced inner diameter portion has a smaller internal diameter than the relatively large inner diameter portion. The relatively reduced inner diameter portion may be positioned distal to the relatively large inner diameter portion. The compliant chemical chamber may extend through at least a length of the relatively reduced inner diameter portion and may also extend through a portion of the length of the relatively large inner diameter portion. The chemical chamber may contain at least the predetermined volume of chemical agent. An opening in the chemical chamber may be in fluid communication with one or more lumens defined by the therapeutic elements. The push member may be attached at a distal end of the push member to a compression element.

In some examples, the compression element includes a flexible ring that surrounds the chemical chamber within the elongate body. A clinician may manipulate the proximal end of the push member (or actuator) to apply a force to the compression element through manipulation (e.g., distal advancing) of the push member, allowing the clinician to advance or retract the flexible ring within the elongate body. When the flexible ring is positioned within the relatively large inner diameter portion, the flexible ring does not apply any force to the compliant walls of the chemical chamber. As the clinician advances the push member distally, the distal end of the push member pushes the flexible ring, and the flexible ring advances distally from the relatively large inner diameter portion to the relatively reduced inner diameter portion. The flexible ring deforms and constricts both its inner and outer diameter as it passes into the relatively reduced inner diameter portion, deforming the compliant wall(s) of the chemical chamber and urging the chemical agent through the opening and through the one or more lumens defined by the therapeutic elements. The relatively reduced inner diameter portion may have a length, such that once a clinician has advanced the flexible ring through the length, the predetermined volume of the chemical agent has been supplied to the treatment site through the therapeutic elements. Although the preceding paragraph describes the ring as “flexible,” in some examples the ring is not flexible, as described above.

In some examples, the compression element includes a ball positioned in the elongate body next to the compliant chemical chamber. A clinician may manipulate the proximal end of the push member (or actuator) to apply a force to the ball through manipulation (e.g., distal advancing) of the push member, allowing the clinician to advance or retract the ball within the elongate body. When the ball is positioned within the relatively large inner diameter portion, the ball does not apply any force to the compliant walls of the chemical chamber. As the clinician advances the push member distally, the distal end of the push member pushes the ball, and the ball advances distally from the relatively large inner diameter portion to the relatively reduced inner diameter portion. The ball may not substantially deform as is passes into the relatively reduced inner diameter portion, pushing against and deforming the compliant wall(s) of the chemical chamber and urging the chemical agent through the opening and through the one or more lumens defined by the therapeutic elements. In some examples, the ball may deform slightly to maintain a smooth transition from the relatively large inner diameter portion to the relatively reduced inner diameter portion. Although the preceding paragraph describes the compression element of as a “ball,” the compression element need not be spherical, and in some examples may include other shapes (e.g., cube, rectangular prism, triangular prism, hemisphere, etc.). The relatively reduced inner diameter portion may have a length, such that once a clinician has advanced the ball through the length, the predetermined volume of the chemical agent has been supplied to the treatment site through the therapeutic elements.

In some examples, the chemical chamber extends through the relatively reduced inner diameter portion and at least a portion of the relatively large inner diameter portion and contains substantially more than the predetermined volume of the chemical agent (i.e., more than one dose of chemical agent). A clinician may advance the compression element of the therapeutic assembly all the way through the relatively reduced inner diameter portion to deliver the predetermined volume of chemical agent to patient tissue at a first treatment site. The clinician may then retract the compression element proximally into the relatively large inner diameter portion. After retracting the compression element, the compression element no longer exerts a force on the compliant chemical chamber, and more chemical agent may flow into or be introduced into the portion of the chemical chamber positioned in the relatively reduced inner diameter portion. In some examples, the interior of the chemical chamber may be in fluid communication with a syringe or other pressurizing mechanism the clinician may use to introduce more chemical agent into the portion of the chemical chamber positioned in the relatively reduced inner diameter portion. In this way, the clinician may navigate the catheter and therapeutic assembly to a second treatment site to supply a second predetermined volume of chemical agent without fully retracting the catheter or therapeutic assembly from the patient's body. The clinician may navigate the catheter and therapeutic assembly to a second treatment site before or after retracting the compression element into the relatively large inner diameter portion.

In some examples, suppling a predetermined volume of chemical agent to tissue includes applying a force on the predetermined volume of chemical agent. The interior of the chemical chamber may be in fluid communication with a syringe or other pressurizing mechanism the clinician may manipulate via the actuator. In some examples, the chemical chamber may extend through the elongate body, and may contain at least the predetermined volume of the chemical agent at a distal portion of the chemical chamber. A plug may be suspended in the chemical agent within the chemical chamber, where the plug is larger than the opening in the chemical chamber, and the plug may travel with the chemical agent toward the opening when a clinician applies a force on the chemical agent. The plug may be located a length away from the opening, such that as the chemical agent is urged through the opening and the therapeutic elements to the treatment site, the plug travels the length and blocks the opening after the predetermined volume of chemical agent has exited the chemical chamber. In some examples, a filler substance is contained within the chemical chamber proximal to the predetermined volume of the chemical agent, defining a boundary between the filler substance and the chemical agent. The filler substance may aid in the transmission of force from the actuator to the chemical agent. The plug may be positioned at the boundary. The clinician may apply force to the filler substance via the actuator (e.g., a syringe), which in turn applies pressure to the predetermined volume of the chemical agent, thereby urging the predetermined volume of the chemical agent through the opening and into the treatment site.

The above detailed descriptions of examples of the technology are not intended to be exhaustive or to limit the technology to the precise form disclosed above. Although specific examples of the technology are described above for illustrative purposes, various equivalent modifications are possible within the scope of the technology, as those skilled in the relevant art will recognize. For example, while steps are presented in a given order, alternative examples may perform steps in a different order. The various examples described herein may also be combined to provide further examples. All references cited herein are incorporated by reference as if fully set forth herein.

From the foregoing, it will be appreciated that specific examples of the present disclosure have been described herein for purposes of illustration, but that various modifications may be made without deviating from the present disclosure. For example, while particular features of the neuromodulation catheters were described as being part of a single device, in other examples, these features can be included on one or more separate devices that can be positioned adjacent to and/or used in tandem with the neuromodulation catheters to perform similar functions to those described herein. Additionally, while the description of the present technology is focused on delivering chemical agents, the present technology can equally be applied to other methods of neuromodulation therapy, including other fluids (e.g., heating, cooling), that may be delivered through direct subcutaneous delivery.

Certain aspects of the present disclosure described in the context of particular examples may be combined or eliminated in other embodiments. Each embodiment and each aspect so defined may be combined with any other embodiment or with any other aspect unless clearly indicated to the contrary. Further, while advantages associated with certain examples have been described in the context of those examples, other examples may also exhibit such advantages, and not all examples need necessarily exhibit such advantages to fall within the scope of the present disclosure. Accordingly, the present disclosure and associated technology can encompass other examples not expressly shown or described herein.

Further, although techniques have been described in which a neuromodulation catheter is positioned at a single location within a single renal artery, in other examples, the neuromodulation catheter may be repositioned to a second treatment site within the single renal artery (e.g., proximal or distal of the first treatment site, may be repositioned in a branch of the single artery, may be repositioned within a different renal vessel on the same side of the patient (e.g., a renal vessel associated with the same kidney of the patient), may be repositioned in a renal vessel on the other side of the patient (e.g., a renal vessel associated with the other kidney of the patient), or any combination thereof. At each location where the neuromodulation catheter is positioned, renal neuromodulation may be performed using any of the techniques described herein or any other suitable renal neuromodulation technique, or any combination thereof.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “about” or “approximately,” when preceding a value, should be interpreted to mean plus or minus 10% of the value, unless otherwise indicated. Additionally, the term “comprising” is used throughout to mean including at least the recited feature(s) such that any greater number of the same feature and/or additional types of other features are not precluded.

Aspects and embodiments of the invention may be defined by the following clauses.

Clause 1. A catheter, comprising:
an elongate body extending from a proximal end to a distal end and comprising a distal portion; and
a therapeutic assembly at the distal portion of the elongate body, wherein the therapeutic assembly comprises a chemical chamber configured to supply a predetermined volume of chemical agent to tissue adjacent the therapeutic assembly.

Clause 2. The catheter of clause 1, wherein one or more walls of the chemical chamber are configured to deform when acted upon by a force or pressure within the distal portion of the elongate body.

Clause 3. The catheter of clause 2, wherein the chemical chamber contains the predetermined volume of chemical agent, and wherein the chemical agent is configured to exit the chemical chamber through an opening in the chemical chamber when the one or more walls of the chemical chamber are acted upon by the force.

Clause 4. The catheter of clause 3, wherein:
the chemical chamber comprises a proximal end and a distal end;
the one or more walls of the chemical chamber comprise at least a proximal wall portion and a distal wall portion;
the proximal wall portion is configured to deform when acted upon by a first force;
the distal wall portion is configured to deform when acted upon by a second force;
the first force is less than the second force.

Clause 5. The catheter of clause 3, wherein:
the chemical chamber comprises a proximal end, a distal end, and a length extending from the proximal end to the distal end;
the one or more walls of the chemical chamber are configured to variably deform along the length.

Clause 6. The catheter of clause 3, wherein the chemical chamber comprises a tube positioned at the distal portion of the elongate body, and wherein the one or more walls of the chemical chamber are configured to compressibly deform when acted upon by the force.

Clause 7. The catheter of clause 3, wherein the chemical chamber comprises:
a tube; and
a separation film defining a first chamber portion and a second chamber portion within the tube, wherein the separation film is configured to deform when acted upon by the force.

Clause 8. The catheter of clause 2, wherein the therapeutic assembly comprises a compression element configured to apply the force to the chemical chamber.

Clause 9. The catheter of clause 8, wherein:
the distal portion of the elongate body comprises a relatively large inner diameter portion and a relatively reduced inner diameter portion, wherein the relatively reduced inner diameter portion is positioned distal to the relatively large inner diameter portion;
the compression element comprises a ball at a distal end of a push member, wherein the ball is positioned in the elongate body next to the chemical chamber; and
the ball is configured to apply the force to the chemical chamber as the ball is pushed distally through the elongate body from the relatively large inner diameter portion and into the relatively reduced inner diameter portion.

Clause 10. The catheter of clause 8, wherein:
the distal portion of the elongate body comprises a relatively large inner diameter portion and a relatively reduced inner diameter portion, wherein the relatively reduced inner diameter portion is positioned distal to the relatively large inner diameter portion;
the compression element comprises a ring at a distal end of a push member positioned in the elongate body around the chemical chamber; and
the ring is configured to apply the force to the chemical chamber as the ring is pushed distally through the elongate body from the relatively large inner diameter portion and into the relatively reduced inner diameter portion.

Clause 11. The catheter of clause 1, wherein:
the chemical chamber contains the predetermined volume of chemical agent,
the chemical agent is configured to exit the chemical chamber through an opening in the chemical chamber when the chemical agent is acted upon by a force;
the therapeutic assembly comprises a plug larger than the opening configured to travel with the chemical agent toward the opening when the chemical agent is acted upon by the force; and
the plug is positioned inside the chemical chamber a length away from the opening such that, as the plug travels along the length toward the opening when the chemical agent is acted upon by the force, the predetermined volume of chemical agent exits the chemical chamber before the plug reaches the opening and prevents more chemical agent from exiting the opening.

Clause 12. The catheter of clause 11, wherein the chemical agent comprises a gel, and wherein the plug is suspended within the gel the length away from the opening.

Clause 13. The catheter of clause 11, wherein the chemical chamber contains a filler substance proximal to the predetermined volume of chemical agent, and wherein the plug is positioned within the chemical agent near a boundary between the filler substance and the predetermined volume of chemical agent.

Clause 14. A method comprising,
positioning a distal portion of an elongate body of a catheter, including a therapeutic assembly, adjacent tissue at a treatment site within a patient;
supplying, via a chemical chamber of the therapeutic assembly, a predetermined volume of chemical agent to the tissue, wherein the chemical chamber contains the predetermined volume of chemical agent.

Clause 15. The method of clause 14, wherein supplying a predetermined volume of chemical agent to the tissue comprises:
applying a force to the chemical chamber of the therapeutic assembly, wherein one or more walls of the chemical chamber are configured to deform when acted upon by the force exerted by a force transmitted within the distal portion of the elongate body, and wherein the predetermined volume of chemical agent is configured to exit the chemical chamber through an opening in the chemical chamber when the one or more walls of the chemical chamber are acted upon by the force.

Clause 16. The method of clause 15, wherein the chemical chamber comprises a tube positioned within the elongate body, and wherein applying the force to the chemical chamber comprises:
pressurizing the elongate body surrounding the tube of the chemical chamber, wherein the tube is configured to compressibly deform when acted upon by the force.

Clause 17. The method of clause 15, wherein:
the chemical chamber comprises:
a tube;
a separation film defining a first chamber portion and a second chamber portion within the tube, wherein the separation film is configured to deform when acted upon by the force, and wherein the second chamber portion contains the predetermined volume of chemical agent; and
applying the force to the chemical chamber comprises pressurizing the first chamber portion within the rigid tube.

Clause 18. The method of clause 15, wherein:
the distal portion of the elongate body comprises a relatively large inner diameter portion and a relatively reduced inner diameter portion, wherein the relatively reduced inner diameter portion is positioned distal to the relatively large inner diameter portion;
the therapeutic assembly comprises a ball at a distal end of a push member positioned in the elongate body next to the chemical chamber; and
applying the force to the chemical chamber comprises advancing the ball and push member distally through the elongate body from the relatively large inner diameter portion and into the relatively reduced inner diameter portion.

Clause 19. The method of clause 15, wherein:
the distal portion of the elongate body comprises a relatively large inner diameter portion and a relatively reduced inner diameter portion, wherein the relatively reduced inner diameter portion is positioned distal to the relatively large inner diameter portion;
the therapeutic assembly comprises a ring at a distal end of a push member positioned in the elongate body around the chemical chamber; and
applying the force to the chemical chamber comprises advancing the ring and push member distally through the elongate body from the relatively large inner diameter portion and into the relatively reduced inner diameter portion.

Clause 20. The method of clause 14, wherein supplying a predetermined volume of chemical agent to the tissue comprises applying a force on the predetermined volume of chemical agent, wherein:
the chemical agent is configured to exit the chemical chamber through an opening in the chemical chamber when the chemical agent is acted upon by the force;
the therapeutic assembly comprises a plug larger than the opening configured to travel with the chemical agent toward the opening when the chemical agent is acted upon by the force; and
the plug is positioned inside the chemical chamber a length away from the opening such that, as the plug travels along the length towards the opening when the chemical agent is acted upon by the force, the predetermined volume of chemical agent exits the chemical chamber before the plug reaches the opening and prevents more chemical agent from exiting the opening.

1. A catheter, comprising:
an elongate body extending from a proximal end to a distal end and comprising a distal portion; and
a therapeutic assembly at the distal portion of the elongate body, wherein the therapeutic assembly comprises a chemical chamber configured to supply a predetermined volume of chemical agent to tissue adjacent the therapeutic assembly.

2. The catheter of claim 1, wherein one or more walls of the chemical chamber are configured to deform when acted upon by a force or pressure within the distal portion of the elongate body.

3. The catheter of claim 2, wherein the chemical chamber contains the predetermined volume of chemical agent, and wherein the chemical agent is configured to exit the chemical chamber through an opening in the chemical chamber when the one or more walls of the chemical chamber are acted upon by the force.

4. The catheter of claim 2, wherein:
the chemical chamber comprises a proximal end and a distal end;
the one or more walls of the chemical chamber comprise at least a proximal wall portion and a distal wall portion;
the proximal wall portion is configured to deform when acted upon by a first force;
the distal wall portion is configured to deform when acted upon by a second force; and
the first force is less than the second force.

5. The catheter of claim 2, wherein:
the chemical chamber comprises a proximal end, a distal end, and a length extending from the proximal end to the distal end; and
the one or more walls of the chemical chamber are configured to variably deform along the length.

6. The catheter of claim 2, wherein the chemical chamber comprises a tube positioned at the distal portion of the elongate body, and wherein the one or more walls of the chemical chamber are configured to compressibly deform when acted upon by the force.

7. The catheter of claim 2, wherein the chemical chamber comprises:
a tube; and
a separation film defining a first chamber portion and a second chamber portion within the tube, wherein the separation film is configured to deform when acted upon by the force.

8. The catheter of claim 2, wherein the therapeutic assembly comprises a compression element configured to apply the force to the chemical chamber.

9. The catheter of claim 8, wherein:
the distal portion of the elongate body comprises a relatively large inner diameter portion and a relatively reduced inner diameter portion, wherein the relatively reduced inner diameter portion is positioned distal to the relatively large inner diameter portion;
the compression element comprises a ball at a distal end of a push member, wherein the ball is positioned in the elongate body next to the chemical chamber; and
the ball is configured to apply the force to the chemical chamber as the ball is pushed distally through the elongate body from the relatively large inner diameter portion and into the relatively reduced inner diameter portion.

10. The catheter of claim 8, wherein:
the distal portion of the elongate body comprises a relatively large inner diameter portion and a relatively reduced inner diameter portion, wherein the relatively reduced inner diameter portion is positioned distal to the relatively large inner diameter portion;
the compression element comprises a ring at a distal end of a push member positioned in the elongate body around the chemical chamber; and
the ring is configured to apply the force to the chemical chamber as the ring is pushed distally through the elongate body from the relatively large inner diameter portion and into the relatively reduced inner diameter portion.

11. The catheter of claim 1, wherein:
the chemical chamber contains the predetermined volume of chemical agent;
the chemical agent is configured to exit the chemical chamber through an opening in the chemical chamber when the chemical agent is acted upon by a force;
the therapeutic assembly comprises a plug larger than the opening configured to travel with the chemical agent toward the opening when the chemical agent is acted upon by the force; and
the plug is positioned inside the chemical chamber a length away from the opening such that, as the plug travels along the length toward the opening when the chemical agent is acted upon by the force, the predetermined volume of chemical agent exits the chemical chamber before the plug reaches the opening and prevents more chemical agent from exiting the opening.

12. The catheter of claim 11, wherein the chemical agent comprises a gel, and wherein the plug is suspended within the gel the length away from the opening.

13. The catheter of claim 11, wherein the chemical chamber contains a filler substance proximal to the predetermined volume of chemical agent, and wherein the plug is positioned within the chemical agent near a boundary between the filler substance and the predetermined volume of chemical agent.

14. A method comprising,
positioning a distal portion of an elongate body of a catheter, including a therapeutic assembly, adjacent tissue at a treatment site within a patient; and
supplying, via a chemical chamber of the therapeutic assembly, a predetermined volume of chemical agent to the tissue, wherein the chemical chamber contains the predetermined volume of chemical agent.

15. The method of claim 14, wherein supplying a predetermined volume of chemical agent to the tissue comprises:
applying a force to the chemical chamber of the therapeutic assembly, wherein one or more walls of the chemical chamber are configured to deform when acted upon by the force exerted by a force transmitted within the distal portion of the elongate body, and wherein the predetermined volume of chemical agent is configured to exit the chemical chamber through an opening in the chemical chamber when the one or more walls of the chemical chamber are acted upon by the force.

16. The method of claim 15, wherein the chemical chamber comprises a tube positioned within the elongate body, and wherein applying the force to the chemical chamber comprises:
pressurizing the elongate body surrounding the tube of the chemical chamber, wherein the tube is configured to compressibly deform when acted upon by the force.

17. The method of claim 15, wherein:
the chemical chamber comprises:
a tube; and
a separation film defining a first chamber portion and a second chamber portion within the tube, wherein the separation film is configured to deform when acted upon by the force, and wherein the second chamber portion contains the predetermined volume of chemical agent; and
applying the force to the chemical chamber comprises pressurizing the first chamber portion within the rigid tube.

18. The method of claim 15, wherein:
the distal portion of the elongate body comprises a relatively large inner diameter portion and a relatively reduced inner diameter portion, wherein the relatively reduced inner diameter portion is positioned distal to the relatively large inner diameter portion;
the therapeutic assembly comprises a ball at a distal end of a push member positioned in the elongate body next to the chemical chamber; and
applying the force to the chemical chamber comprises advancing the ball and push member distally through the elongate body from the relatively large inner diameter portion and into the relatively reduced inner diameter portion.

19. The method of claim 15, wherein:
the distal portion of the elongate body comprises a relatively large inner diameter portion and a relatively reduced inner diameter portion, wherein the relatively reduced inner diameter portion is positioned distal to the relatively large inner diameter portion;
the therapeutic assembly comprises a ring at a distal end of a push member positioned in the elongate body around the chemical chamber; and
applying the force to the chemical chamber comprises advancing the ring and push member distally through the elongate body from the relatively large inner diameter portion and into the relatively reduced inner diameter portion.

20. The method of claim 14, wherein supplying a predetermined volume of chemical agent to the tissue comprises applying a force on the predetermined volume of chemical agent, wherein:
the chemical agent is configured to exit the chemical chamber through an opening in the chemical chamber when the chemical agent is acted upon by the force;
the therapeutic assembly comprises a plug larger than the opening configured to travel with the chemical agent toward the opening when the chemical agent is acted upon by the force; and
the plug is positioned inside the chemical chamber a length away from the opening such that, as the plug travels along the length towards the opening when the chemical agent is acted upon by the force, the predetermined volume of chemical agent exits the chemical chamber before the plug reaches the opening and prevents more chemical agent from exiting the opening.
